SEQUENCE LISTING (14 sequences)

SEQ ID NO: 1 - DNA, 1429 bp, Mus musculus
  misc_feature (1)..(1429): mouse p53
ttcctggnctgtaggtagcgactacagttagggggcacctagcattcaggccctcatcctcctccttcccagcagggtgtcacgcttctccgaagactggatgactgccatggaggagtcacagtcggatatcagcctcgagctccctctgagccaggagacattttcaggcttatggaaactacttcctccagaagatatcctgccatcacctcactgcatggacgatctgttgctgccccaggatgttgaggagttttttgaaggcccaagtgaagccctccgagtgtcaggagctcctgcagcacaggaccctgtcaccgagacccctgggccagtggcccctgccccagccactccatggcccctgtcatcttttgtcccttctcaaaaaacttaccagggcaactatggcttccacctgggcttcctgcagtctgggacagccaagtctgttatgtgcacgtactctcctcccctcaataagctattctgccagctggcgaagacgtgccctgtgcagttgtgggtcagcgccacacctccagctgggagccgtgtccgcgccatggccatctacaagaagtcacagcacatgacggaggtcgtgagacgctgcccccaccatgagcgctgctccgatggtgatggcctggctcctccccagcatcgtatccgggtggaaggaaatttgtatcccgagtatctggaagacaggcagacttttcgccacagcgtggtggtaccttatgagccacccgaggccggctctgagtataccaccatccactacaagtacatgtgtaatagctcctgcatggggggcatgaaccgccgacctatccttaccatcatcacactggaagactccagtgggaaccttctgggacgggacagctttgaggttcgtgtttgtgcctgccctgggagagaccgccgtacagaagaagaaaatttccgcaaaaaggaagtcctttgccctgaactgcccccagggagcgcaaagagagcgctgcccacctgcacaagcgcctctcccccgcaaaagaaaaaaccacttgatggagagtatttcaccctcaagatccgcgggcgtaaacgcttcgagatgttccgggagctgaatgaggccttagagttaaaggatgcccatgctacagaggagtctggagacagcagggctcactccagctacctgaagaccaagaagggccagtctacttcccgccataaaaaaacaatggtcaagaaagtggggcctgactcagactgactgcctctgcatcccgtccccatcaccagcctccccctctccttgctgtcttatgacttcagggctgagacacaatccccaggatccactcanactgnctgcctntgnatccngtccccatnaccancctccccntntccttgctgnnttatgact

SEQ ID NO: 2 - DNA, 1515 bp, Mus musculus
  misc_feature (1)..(1515): mouse Pten
ccacgcgtccgcggacgcgtgggcggacgcgtgggcccacgcgtccggccgccgcagcagccattacccggctgcggtccagggccaagcggcagcagagcgaggggcatcagcgaccgccaagtccagagccatttccatcctgcagaagaagcctcgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaatattattgctatgggatttcctgcagaaagacttgaaggtgtatacaggaacaatattgatgatgtagtaaggtttttggattcaaagcataaaaaccattacaagatatacaatctatgtgctgagagacattatgacaccgccaaatttaactgcagagttgcacagtatccttttgaagaccataacccaccacagctagaacttatcaaacccttctgtgaagatcttgaccaatggctaagtgaagatgacaatcatgttgcagcaattcactgtaaagctggaaagggacggactggtgtaatgatttgtgcatatttattgcatcggggcaaatttttaaaggcacaagaggccctagatttttatggggaagtaaggaccagagacaaaaagggagtcacaattcccagtcagaggcgctatgtatattattatagctacctgctaaaaaatcacctggattacagacccgtggcactgctgtttcacaagatgatgtttgaaactattccaatgttcagtggcggaacttgcaatcctcagtttgtggtctgccagctaaaggtgaagatatattcctccaattcaggacccacgcggcgggaggacaagttcatgtactttgagttccctcagccattgcctgtgtgtggtgatatcaaagtagagttcttccacaaacagaacaagatgctcaaaaaggacaaaatgtttcacttttgggtaaatacgttcttcataccaggaccagaggaaacctcagaaaaagtggaaaatggaagtctttgtgatcaggaaatcgatagcatttgcagtatagagcgtgcagataatgacaaggagtatcttgtactcaccctaacaaaaaacgatcttgacaaagcaaacaaagacaaggccaaccgatacttctctccaaattttaaggtgaaactatactttacaaaaacagtagaggagccatcaaatccagaggctagcagttcaacttctgtgactccagatgttagtgacaatgaacctgatcattatagatattctgacaccactgactctgatccagagaatgaaccttttgatgaagatcagcattcacaaattacaaaagtctgatttttttttttcttatcaagagggataaaataccatgaaaaaaaaaacttgaataaactgaaatggacctttaaaaaaaaaaaaaa

SEQ ID NO: 3 - DNA, 22 bp, Artificial
  Synthetic oligo
cacaaaaacaggttaaacccag

SEQ ID NO: 4 - DNA, 20 bp, Artificial
  Synthetic oligo
agcacataggaggcagagac

SEQ ID NO: 5 - DNA, 19 bp, Artificial
  Synthetic oligo
actcaaggcagggatgagc

SEQ ID NO: 6 - DNA, 22 bp, Artificial
  Synthetic Oligo
gtcatcttcacttagccattgg

SEQ ID NO: 7 - DNA, 17 bp, Artificial
  Synthetic oligo
ggcgtgtgccatcaatg

SEQ ID NO: 8 - DNA, 17 bp, Artificial
  Synthetic oligo
aactcaagggagacctg

SEQ ID NO: 9 - DNA, 21 bp, Artificial
  Synthetic oligo
gtcgacaagctcatgcgggtg

SEQ ID NO: 10 - DNA, 27 bp, Artificial
  Synthetic oligo
cctttacaagcgcacgcagactgtaga

SEQ ID NO: 11 - DNA, 31 bp, Artificial
  Synthetic oligo
agctagccaccatggcttgagtaagtctgca

SEQ ID NO: 12 - DNA, 21 bp, Artificial
  Synthetic oligo
gcgaagagtttgtcctcaacc

SEQ ID NO: 13 - DNA, 21 bp, Artificial
  Synthetic oligo
ggagcgggagaaatggatatg

SEQ ID NO: 14 - DNA, 21 bp, Artificial
  Synthetic oligo
aaagtcgctctgagttgttat

(19) United States
(12) Patent Application Publication
(10) Pub. No.: US 2008/0216180 A1
(43) Pub. Date: Sep. 4, 2008

(54) Animal model for cancer, methods of producing the same and associated methods of use

(76) Inventors: Corrine T. Abate-Shen, Warren, NJ (US); Anna Puzio-Kuter, East Brunswick, NJ (US)

Correspondence Address: MCCARTER & ENGLISH LLP;CITYPLACE I, 185 ASYLUM STREET, HARTFORD, CT, 06103, US

(21) Appl. No.: 11/888,014

(22) Filed: Jul. 30, 2007

Related U.S. Application Data
(60) Provisional application No. 60/834,013, filed on Jul. 28, 2006.

(51) Int. Cl. G01N 33/53; A01K 67/027; C12N 15/85; C12Q 1/68

(52) U.S. Cl. 800/3; 800/8; 800/18; 800/21; 435/6

(57) ABSTRACT

A transgenic animal having a somatic cell in which at least one allele of an endogenous p53 and Pten gene is functionally disrupted is provided. The cell of the animal may be heterozygous or, more preferably, homozygous for the gene disruptions. The animals of the invention can be used to evaluate the efficacy of novel therapeutics and to identify novel points of therapeutic intervention for cancer. In certain embodiments, the transgenic animal is a transgenic mouse having functionally disrupted endogenous p53 and Pten genes. This mouse can be used to identify agents that inhibit the development of cancers, namely bladder cancers in humans in vivo. Nucleic acid constructs for functionally disrupting an endogenous p53 and Pten gene in a host cell, recombinant vectors including the nucleic acid construct, and host cells into which the nucleic acid construct has been introduced are also encompassed by the invention

### CROSS-REFERENCE TO RELATED APPLICATIONS

Under 35 U.S.C. § 119(e) this application claims the benefit of U.S. Provisional Application No. 60/834,013 filed Jul. 28, 2006, which is hereby incorporated by reference in its entirety.

### INCORPORATION BY REFERENCE

In compliance with 37 C.F.R. § 1.52(e)(5), the sequence information contained on compact disc, file name: Abate-Shen_{—}2007 utility_ST25.txt; size 8 KB; created on: 30 Jul. 2007; using PatentIn-3.4, and Checker 4.4.0 is hereby incorporated by reference in its entirety. The Sequence Listing information recorded in computer readable form (CRF) is identical to the written Sequence Listing provided herewith. The data in the paper copy of the Sequence Listing, and Computer Readable Form of the Sequence Listing submitted herewith contain no new matter, and are fully supported by the priority application, U.S. Provisional Patent Application No. 60/834,013.

### STATEMENT REGARDING FEDERALLY SPONSORED RESEARCH

The U.S. Government has certain rights in this invention pursuant to Grant No.: U01 CA084294 (Apr. 1, 2004-Mar. 31, 2009), entitled: A Mouse Model for Prostate Cancer; to Corrine T. Abate-Shen, awarded by the National Institutes of Health (NIH), National Cancer Institute.

### FIELD OF THE INVENTION

The present disclosure describes an animal model for studying the role of specific genes in the development of cancer, and the use of such models for screening for novel therapeutics and therapeutic interventions.

### BACKGROUND

Despite significant improvements in diagnosis and treatment, neoplasia of the bladder continues to result in significant mortality. Bladder cancers range from being benign to highly aggressive, and most are epithelial in origin and urothelial in nature. They are believed to arise from two distinct precursor lesions, namely, a papillary form (called Papillary Urothelial Neoplasm of Low Malignant Potential or PUNLMP) and carcinoma-in-situ (called CIS).

However, it is currently not well understood how these precursors relate to each other or to benign versus invasive disease.

### SUMMARY OF THE INVENTION

To investigate the origin and underlying molecular pathways of cancer an animal model is provided that has a disruption in the endogenous genes for p53 and Pten (See, for example SEQ ID NOs: 1 and 2, respectively). In certain aspects, the invention relates to the creation of an autochthonous animal model for the study of bladder neoplasia, oncogenesis, and metastasis comprising a disruption in the endogenous genes for p53 and Pten. In one embodiment, the transgenic animal having a disruption in the p53 and Pten genes, and demonstrating a bladder cancer phenotype is a mouse. The transgenic mouse provided herein presents a cancer pathology that originates in the bladder urothelium and ultimately progresses to invasive disease.

Although the present specification describes a double knockout mouse model for bladder cancer. It will be appreciated by those of skill in the art that the invention is not limited to any particular animal species. For example, due to the fact that the p53 and Pten genes are conserved across species, the methods and techniques described herein can be easily modified according the present teachings to create a animal cancer model from other suitable animal species commonly used in biomedical research, including for example, dog, pig, sheep, rat, and the like.

In additional aspects, the invention relates to methods of using the transgenic animal of the invention and/or cells derived therefrom for the evaluation of the potential therapeutic candidates for the treatment of cancer, in particular, bladder cancer. In addition, the invention encompasses methods of using the transgenic animal of the invention and/or cells derived therefrom for the identification of novel points of therapeutic intervention for the treatment of cancer, in particular, bladder cancer.

In further aspects, the invention relates to nucleic acid constructs for functionally disrupting an endogenous p53 and Pten gene in a host cell, recombinant vectors including the nucleic acid construct, and host cells into which the nucleic acid construct has been introduced are also encompassed by the invention.

In a further aspect, the invention relates to cancer cells isolated from the transgenic animal of the invention. In another embodiment, the invention relates to methods for screening potential therapeutic agents, in vitro, comprising contacting a compound to the cancerous cells isolated from the transgenic animal of the invention. In still other embodiments, the invention relates to methods of performing ex vivo immunotherapy comprising isolating immune cells, for example, T cells, dendritic cells, or APC cells, from a host and exposing them to cancer cells isolated from a transgenic animal of the invention, and subsequently administering the activated immune cells back to the host.

Additional objects and advantages of the present invention will be appreciated by one of ordinary skill in the art in light of the instant drawings, detailed description, examples, and claims. These additional objects and advantages are expressly included within the scope of the present invention.

### BRIEF DESCRIPTION OF THE DRAWINGS

FIG. 1: Diagram of human bladder with inset of different uroepithelial layers.

FIG. 2: Diagram to show the histology of the transitional epithelium in the contracted bladder, A, and in the dialated bladder, B, of the rat. C=blood capillaries, M=muscle fibers, b.l.=basal lamina, B=basal layer, I=intermediate layer, S=superficial layer, g=granules, l.m.=luminal membrane, f=fusiform vacuoles, n=binucleated. (Adapted from Hicks 1975).

FIG. 3: Injection strategy of Ad-cre into the bladder lumen.

FIG. 4: Ki-67 and adenovirus targeted cells are likely in the same epithelial layer.

FIG. 5: A, Gross anatomy of bladder metastases to the intestinal lining. B, hematoxylin and eosin staining and cytokeratin 7 staining in adjacent sections of liver metastases.

FIG. 6: Survival Curve. A, Kaplan Meier survival curve of control, single, and double mutants. Results are statictically significant, P<0.0001. B, Kaplan Meier survival curve of control, p53^{LoxP/Loxp}/Rb^{LoxP/Loxp}, and p53^{LoxP/Loxp}/Pten^{LoxP/LoxP} double mutant mice. Results are statistically significant, P<0.00001.

FIG. 7: Gross anatomy of control, single mutant, and double mutant mice with average bladder/bladder tumor weights. Note: P53^{LoxP/LoxP}; Pten^{LoxP/LoxP} are for mice dissected 2.5-6 months after injection.

FIG. 8: Histological analysis of mouse and human invasive bladder tumors using hematoxylin and eosin staining.

FIG. 9: Immunohistochemical analysis of control, single and double mutant mice showing hematoxylin and eosin staining, broad cytokeratin staining, and Ki-67 proliferation marker.

FIG. 10: Terminal transferase dUTP nick end labeling (TUNEL) assay of control and double mutant mice.

FIG. 11: Immunohistochemical staining of control, single and double mutant mice.

FIG. 12: Analysis of apoptotic cells in control, single and double mutant mice using a TUNEL assay.

FIG. 13: Western analysis of p53 and Pten proteins in normal bladder and p53^{LoxP/LoxP}; Pten^{LoxP/LoxP} tumors show both proteins are down in expression as compared to normal bladder.

FIG. 14: A, p19^{ARF} expression is nucleolar as compared to PML and filbrillar. B, p19^{ARF} expression in control, single and double mutants showing increased expression in the tumors of p53^{LoxP/LoxP}; Pten^{LoxP/LoxP} mice.

FIG. 15: A, tissue recombinants using embryonic rat 15.5 dpc mesenchyme and adult rat epithelium. B, tissue recombinants using embryonic rat 15.5 dpc mesenchyme and human RT4 bladder cells.

FIG. 16: Plasmid map of MSCV-LTR miR30-PIG (MSCV-LMP) retroviral vector used for expression of shRNA.

### DETAILED DESCRIPTION OF THE INVENTION

While the specification concludes with claims particularly pointing out and distinctly claiming the subject matter regarded as forming the present invention, it is believed that the invention will be better understood from the following preferred embodiments of the invention taken in conjunction with the accompanying drawings.

The present invention is based on the discovery that an animal model comprising the double knockout of at least one allele of p53 and Pten genes results in the creation of an autochthonous animal model for the study of bladder neoplasia, oncogenesis, and metastasis. In one embodiment, the transgenic animal having a disruption in the p53 and Pten genes, and demonstrating a bladder cancer phenotype is a mouse. The transgenic mouse provided herein presents a cancer pathology that originates in the bladder urothelium and ultimately progresses to invasive disease.

Mouse models have been used to understand various developmental stages and disease that otherwise would not be able to be studied in humans. Bladder cancer is devastating in that muscle-invasive disease comes with a poor prognosis if detected in late stages, and the non-invasive disease is treatable but with continued monitoring for the rest of the patient's life. The two pathways of bladder cancer, non-invasive and muscle-invasive, are thought to occur via different genetic alterations. Therefore it is important to design new models of bladder cancer that could recapitulate various stages of the disease in order to understand the genetics and molecular biology of the disease.

Using a conditional approach, we generated mice with different combinations of tumor suppressor and oncogenes in order in investigate the consequences of loss of function or activation, respectively. In doing so, coopertivity between p53 and Pten was discovered to be important in muscle-invasive bladder cancer, where the bladder tumors that arose in these mice were histologically similar and progressed like the human counterparts.

In another aspect, the present invention features a nucleic acid molecule, such as a decoy RNA, dsRNA, siRNA, shRNA, micro RNA, aptamers, antisense nucleic acid molecules, which down regulates expression of a sequence encoding p53 and Pten. In an embodiment, a nucleic acid molecule of the invention is adapted to treat bladder disorders, for example, bladder cancer. In another embodiment, a nucleic acid molecule of the invention has an endonuclease activity or is a component of a nuclease complex, and cleaves RNA having a p53 or Pten nucleic acid sequence.

In one embodiment, a nucleic acid molecule of the invention comprises between 12 and 100 bases complementary to RNA having a p53 or Pten nucleic acid sequence. In another embodiment, a nucleic acid molecule of the invention comprises between 14 and 24 bases complementary to RNA having a p53 or Pten nucleic acid sequence. In any embodiment described herein, the nucleic acid molecule can be synthesized chemically according to methods well known in the art.

In another aspect, the invention relates to a method for diagnosing or monitoring a bladder disorder disease or progression comprising detecting for the presence of a nucleotide polymorphism in the p53 or Pten structural gene associated with bladder disease or disease severity, through the detection of the expression level of a p53 or Pten gene or protein or both. As used herein, “gene” or “structural gene” includes the 5′ UTR, 3′ UTR, promoter sequences, enhancer sequences, intronic and exonic DNA of the MIF gene as well as the MIF gene mRNA or cDNA sequence.

As one of ordinary skill will comprehend, the gene polymorphisms associated with bladder disorders and hence useful as diagnostic markers according to the methods of the invention may appear in any of the previously named nucleic acid regions. Techniques for the identification and monitoring of polymorphisms are known in the art and are discussed in detail in U.S. Pat. No. 6,905,827 to Wohlgemuth, which is incorporated herein by reference in its entirety for all purposes.

Certain aspects of the invention encompass methods of detecting gene expression or polymorphisms with one or more DNA molecules wherein the one or more DNA molecules has a nucleotide sequence which detects expression of a gene. In one format, the oligonucleotide detects expression of a gene that is differentially expressed. The gene expression system may be a candidate library, a diagnostic agent, a diagnostic oligonucleotide set or a diagnostic probe set. The DNA molecules may be genomic DNA, RNA, protein nucleic acid (PNA), cDNA or synthetic oligonucleotides. Following the procedures taught herein, one can identify sequences of interest for analyzing gene expression or polymorphisms. Such sequences may be predictive of a disease state.

The bladder originates from the endoderm and grows during development through mesenchymal and epithelial interactions whose simple organization comes with a very important function, the retention and voiding of urine. Bladder cancer occurs though two molecular pathways where one leads to a papillary non-invasive disease which is relatively manageable and the other to a muscle invasive disease whose prognosis is very poor. Understanding pathways associated with chromosomal, genetic and epigenetic alterations are important for deciphering molecular pathway changes as well as for the design of targeted therapeutics.

The primary function of the mammalian bladder is to retain urine produced by the kidney though not regulate its composition until it can be voided by the body. The urine that enters the bladder is mostly hypertonic and is kept there by the unique barrier feature of the uroepithelium. This barrier protects from loss of endogenous water that would result in dehydration. Artificially constructed bladders from the ileal or rectum result in resorption of electrolytes and nitrogenous wastes resulting in such complications as uraemia. Voiding involves the unique arrangement of the musculature wall of the bladder, which allows the bladder to expand and contract in response to urine volume without an increase in internal pressure. Finally, closing of the ureteral orifices allows for the urine to flow into the urethra and out of the body.

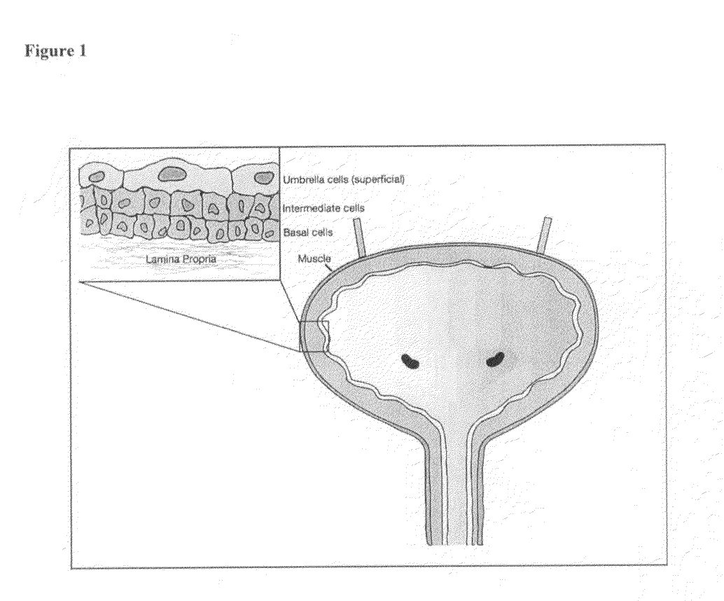

The mammalian bladder has three main layers; serous, muscular and mucosa. The serous layer is composed of connective tissue with a thin layer of mesothelial cells. The muscular wall is usually composed of three layers of smooth muscle, two longitudinal and one central circular muscle. The mucosal layer closest to the muscle layer is the lamina propria, which is a fibrous connective tissue that carries blood vessels and nerve fibres, followed by an uninterrupted basal lamina below basal epithelial cells. The epithelium (uroepithelium), may be anywhere from three to twelve layers and depends first on the organism and secondly on the filling or voiding of the bladder where a full bladder has a reduced number of cells layers to accommodate the relaxing muscle layer. The uroepithelial cells exhibit a regular pattern of differentiation having undifferentiated cells at the base all the way to highly differentiated cells at the surface of the epithelium (FIG. 1). The basal cells are a single layer that is in direct contact with the underlying connective tissue and capillary bed and serve as precursors to the other cell layers, while the intermediate cells sit on top of the underlying basal cells and can be anywhere from one to several layers thick.

The layer that is closest to the lumen is that of the umbrella cells that is comprised of very large polyhedral cells and can be multinucleated and even can be polyploidal in some species. This is suggested to occur by intermediate cell-cell fusion with retention of individual nuclei (multi-nucleated) or results in nuclei fusion (poly-ploidy). The apical surface of umbrella cells under high magnification has a pleated look with each cell surrounded by a tight junctional ring. This pleated look is due to raised ridges, also known as hinges, and plaques, which are the areas in between though the exact function of this cellular morphology is not completely understood.

Uroepithelial function. The primary function of the uroepithelium is to form a barrier against pathogens, and to keep a tight control on the passage of water, ions, solutes, and large macromolecules across the mucosal surface of bladder. This barrier is highly effective in the bladder due to the presence of tight junctions that form a type of seal in the umbrella cell layer, and thus splits the membrane of an umbrella cell into apical and basolateral domains.

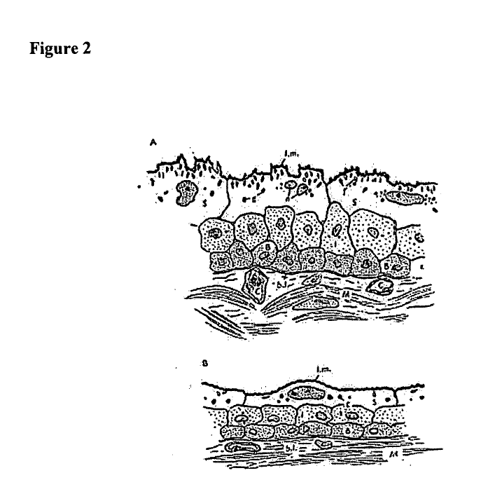

The uroepithelium undergoes cyclical changes in hydrostatic pressure in response to filling and voiding and during this process must maintain the barrier function, which is also accompanied by the unfolding (bladder filling) and folding (bladder voiding) of the mucosal surface and morphological changes of the uroepithelium (FIG. 2). Specifically, intermediate and basal cells are pushed laterally and the umbrella cells undergo a shape change from a roughly cuboidal morphology (empty bladder) to one that is flat and squamous (filled bladder). Also the umbrella cells are able to undergo this shape change by discoidal/fusiform vesicle exocytosis that increases the apical surface and the overall surface area in the bladder lumen. In contrast, during bladder voiding, the added apical membrane is internalized by endocytosis and reestablishes the vesicle pool.

Uroepithelial histology. The uroepithelium is a specialized tissue with differentiated superficial cells on the surface closest to the lumen. The basal cells are cuboidal or columnar and their cytoplasm is strongly basophilic where mitotic figures are very seldom seen and are attached to the underlying basal lamina by desmosomes at frequent intervals. The intermediate cells contain lots of lysosomes and their cytoplasm is less basophilic. Their lateral membranes can be folded in response to voiding of the bladder and are kept attached to neighboring cells by desmosomes. The epithelial cells contacting the urinary surface are large, flat squamous cells that are basophilic and contain large lysosomes.

Bladder Cancer—Transitional Cell Carcinoma. Bladder cancer is the fifth most common cancer in the United States with an estimated 63,000 new cases every year with a majority of those case being patients of middle and old age with a male:female ratio of ˜3:1. There are three main types of cancers that affect the bladder; uroepithelial carcinoma (transitional cell carcinoma, TCC), squamous cell carcinoma, and adenocarcinoma, with the majority of cases being transitional cell carcinomas. Smoking has been shown to be the major contributor of the disease though others such as occupational carcinogens, bladder stones, chronic indwelling Foley catheters, schistosomiasis, and chemical irritations have also been shown to be risk factors. Most epithelial cancers are thought to progress via a single pathway, however in epithelial bladder cancer, scientific evidence points to two distinct mechanisms. The mechanisms are thought to be due to specific genetic alterations, which have lead to the analysis of the two main phenotypic variants, low grade papillary and invasive transitional cell carcinoma each with their molecular pathways, prognoses, and treatments.

The low-grade papillary type accounts for approximately 80% of urothelial carcinomas and is thought to be originated from simple and nodular urothelial hyperplasia and branching vasculature. Such tumors are usually well differentiated and do not penetrate the epithelial basement membrane or are associated with high-grade lesion carcinoma in situ (CIS) and thus are considered stage Ta. If detected early, these tumors are treated with surgical resection and intravesical (within the bladder) immunotherapy and patients are then monitored for recurrence at regular intervals by cytoscopic observation with a good 5 year survival rate of 90%.

Approximately 20% of urothelial carcinomas do not display a previous history of papillary tumors, are highly aggressive, invade into the muscle layer and metastasis to local organs. Metastasis is one of the major problems of this disease since 50% of patients diagnosed with metastatic invasive bladder carcinoma die within 2 years. These tumors are usually poorly differentiated and are either associated with CIS, which is thought to be the precursor lesion of this type of tumor, or seem to arise do novo. Currently, treatment opinions include radical cystectomy and debilitating systemic therapy.

Stages of bladder cancer. Staging of bladder cancer is based on the Tumor, Node, Metastasis system (TNM system) where superficial (noninvasive) transitional cell carcinoma ranges from stages Tᵢₛ to T₁ while muscle invasive tumors range from T₂ to T_{4b}. The definitions of the stages are as follows: T is and carcinoma-in-situ (CIS) are restricted to the mucosa with no lamina propria invasion, T₁ tumors have penetrated the lamina propria, T₂ are superficial muscle invasive tumors, T₃ are deep muscle invasive tumors and T_{3b} tumors have further invaded the perivesical fat, T₄ₐ represents prostate or vagina invasion, and T_{4b} are tumors that are fixed to the pelvis or abdominal wall. Other grading involves that of the lymph nodes (N₁ to N₄), distant metastasis (M₀ to M₁) and histological characteristics (G₁, G₂ or G₃).

Molecular pathways involved in bladder cancer. Cancer can be thought of as natural processes gone terribly wrong. Normal cellular proliferation goes through a specific progression of events through the cell cycle and even if there is a “problem” there are checkpoints to allow the cells to repair themselves before being allowed to potential pass on the mutation. There are several genes that are involved in the cell cycle with mutation or inactivation of any of them leading to loss of control and ultimately progression in carcinogenesis. The inactivation of both copies of a gene can occur by: (1) primary alteration of one copy followed by a second somatic “hit” or (2) two somatic “hits” in both copies of gene. Historically, individual genes were studied to understand their role in the cell cycle however, recently the times have changed and it is now believed that whole pathways and the interactions of multiple tumor suppressor genes are involved in the progression of bladder cancer.

p53 tumor suppressor gene. The p53 protein was first detected in complexes with the SV40 T antigen almost 3 decades ago. The cDNA was then cloned from SV40-transformed cells and was first considered to be an oncogene that could collaborate with Ras to transform primary rodent cells. It wasn't until a few years later that p53 was shown to act as a suppressor of transformation which has led to the unraveling of its complex functions as a transcription factor activated in response to cellular stress or oncogene activation, as a regulator of cell proliferation, an inducer of apoptosis, and a tumor suppressor. The origin of p53 mutations as well as the mutational spectrum in different human cancers has been well studied. In bladder cancer, p53 mutations are correlated with its overexpression, similarly, 17p LOH is also associated with its nuclear overexpression.

It has been suggested that p53 does not have a role in the origin of bladder cancer (Hartmann et al., 1999). Tissue from biopsies of patients with urothelial hyperplasias and simultaneous or consecutive superficial papillary tumors were analyzed at chromosomes 9 and 17 for deletions (chromosomal location of p53 is 17p13). Ten out of 14 hyperplasies (70%) showed a deletion at chromosome 9 but none of them showed deletions at chromosome 17.

However, nuclear p53 detection was associated with an increased risk of recurrence of bladder cancer (P<0.001) in histological specimens of transitional cell carcinoma of the bladder from patients treated by radical cystectomy (Esrig et al., 1994). The rates of recurrence for stage P1, P2, and P3a tumors with p53 immunoreactivity was 62, 56 and 80%, respectively.

p53 is associated with many different proteins depending on the cellular process and those associations can be disrupted if p53 or another protein in the corresponding pathway is mutated. The p53 protein is a transcription factor that is regulated by Mdm2, which binds to it and keeps it from activating its target genes such as those involved in apoptosis, DNA damage and cell cycle arrest (p21/WAF1). Specifically looking at the p53 pathway components and their relevance in bladder cancer reveals the impact of alterations in key genes such as Mdm2 and p21. Tumor samples from 140 patients were used to first identify mutations in the p53 gene, which occurred in 79 cases (56.4%) confirming the importance of p53 in bladder cancer. The mutations included: by direct sequencing 66 individual points mutations and five frameshifts and by oligonucleotide array 65 mutations and four slice site mutations. Illustration of the importance of the different components of the p53 pathway by immunohistochemistry revealed that individually mdm2 overexpression or p21 loss was of no clinical significance but each in combination with altered p53 status had decreased survival. The accumulation of defects such as a mutant TP53 and/or p53 overexpression, loss of p21 nuclear expression and mdm2 nuclear overexpression indicated the worst clinical outcome and patient survival and represents a significant predictive factor. However, p53 does not work in all the pathways that lead to bladder cancer and since this is the case alternative pathways must be identified to understand the pathogenesis of those tumors (p53 wildtype).

FGFR3 and p53 have been found to be involved in two distinct pathways leading to urothelial cell carcinoma. FGFR3 is a tyrosine kinase receptor that regulates cell growth, differentiation, migration, wound healing and angiogenesis and when mutated is responsible for skeletal abnormalities as well as carcinoma. Studying the concurrence of p53 and FGFR3 mutations in primary urothelial cell carcinomas revealed that they were almost mutual exclusive events. FGFR3 mutations occurred in samples that were low stage/low grade and had a favorable outcome while p53 mutations were associated with high stage/high grade tumor samples. These results agree with observations supporting two alternative pathways though in that model alterations in chromosome 9 were important in superficial papillary tumors, and TP53 mutations were frequent in CIS and invasive tumors (Spruck et al., 1994), though from the aforementioned studies it is unclear where LOH of chromosome 9 fits into the progression of superficial papillary tumors with FGFR3 mutations.

Pten tumor suppressor gene. Pten/MMAC1 was identified as a tumor suppressor gene located on chromosome 10q23 responsible for Cowden disease, which is an autosomal dominant cancer predisposing syndrome. PTEN plays important roles in many cellular processes such as cell cycle arrest, programmed apoptosis, cell physiology, regulation of cell adhesion, migration and differentiation. PTEN contains a protein tyrosine phosphatase (PTP) domain that is able to dephosphorylate both tyrosine and serine/threonine residues with its main substrate being phosphatidylinositol (3,4,5)-triphosphate (PIP-3). PIP-3 accumulation recruits proto-oncogene serine/threonine kinase, Akt, which allows for its phosphorylation and subsequent activity in cell cycle control and inactivation of apoptosis. Thus in cancer a loss of PTEN results in elevated PIP-3 levels, Akt hyperactivation, and protection from apoptotic stimulation.

In several analyses, PTEN was shown to have several types of alterations including somatic point mutations, frameshifts, splicing variants, and homozygous deletions that were associated with late-stage and invasive bladder cancers (superficial tumors 6.6%), though frequency of mutations differed from low to only slightly higher. Similarly, a decrease in PTEN expression also correlated with muscle invasive TCC, and even further protein expression reduction was found from primary tumor to corresponding metastasis. The mechanism of inactivation of the second allele of the PTEN gene in the progression of bladder cancer was suggested to be most likely due to homozygous deletion verses small sequence alterations. Though overall functional loss of PTEN may be higher than detected by LOH or gene alterations as in the above studies through translational modification or promoter hypermethylation.

With PTEN being genetically altered in muscle invasive bladder, there must be some selective pressure for deletion of its tumor suppression function. Simple introduction of exogenous PTEN via an adenoviral vector into UM-UC-3 and T24 cells lines (PTEN deficient) resulted in suppressed tumor growth and arrest in the G1 phase of the cell cycle. Similarly, restoration of a dominant negative version of the PI-3 kinase target, Akt, in the same cell line (UM-UC-3) also inhibited invasion as analyzed by an in vitro invasion assay. Further investigation into the mechanism through which PTEN exerts its tumor suppressive function, examined its lipid phosphatase activity. Stably transfected bladder cancer cell lines with wild-type, G129E mutant (lipid phosphatase deficient), or G129R (protein and lipid phosphatase deficient) were used in a organotypic in vitro invasion assay to show that wild-type and the G129E mutant were able to block invasion and inhibit chemotaxis in vitro, while the G129R mutant was unable. This illustrated that Pten's tumor suppressive mechanisms were independent of its lipid phosphatase activity.

In vivo models of bladder cancer. Animal models have been used to unravel many aspects of carcinogenesis, which include initiation, promotion and progression. To study bladder cancer, there has been great strides taken to create transgenic mice with promoter-driven gene expression that is specific to the bladder urothelium (Garcia-Espana et al., 2005; Klein et al., 2005; Lin et al., 1995; Tsuruta et al., 2006; Yoo et al., 2006; Zhang et al., 2001; Zhang et al., 1999). Transgenic mice with Keratin 5 promoter driven cyclooxygenase-2 (COX-2) were prone to transitional cell hyperplasia in heterozygous (17%) and homozygous (75%) mice and transitional cell carcinoma in ˜10% of mice, indicating a role for this gene in bladder cancer. Illustrating roles for other genes came from the use of an urothelium-specific promoter of the uroplakin II gene. Transgenic mice with urothelial over-expression of Cyclin-D1 showed no morphologic abnormality but did have increased levels of p21 and p27 cell cycle inhibitors, and expression of the SV40T antigen, which inactivates p53 and pRb, induced carcinoma in situ and invasive and metastatic bladder cancer. In striking contrast, the urothelial expression of an activated Ha-ras caused urothelial hyperplasia and superficial papillary non-invasive bladder tumors providing evidence that the two phenotypical pathways of bladder tumorigenesis are caused by distinctive genetic defects.

More recently, conditional knock-out mice using the Cre-loxP system, allowed the generation of Fabpl-cre:Pten^{loxP/loxP} mice that allow for Pten to be deficient in the bladder urothelium (as well as the intestinal epithelium, uroepithelium of the kidney and ureter, prostate, seminal vesicle) (Tsuruta et al., 2006; Yoo et al., 2006). These mice exhibited urothelial hyperplasia accompanied with enlarged nuclei and increased cell size and in 10% of 10-20 month old homozygote animals (Fabpl-cre^{cre/+}:Pten^{loxP/loxP}) and homozygote animals treated with the chemical carcinogen N-butyl-N-(4-hydroxybutyl) nitrosamine there was development of papillary transitional cell carcinoma that showed increased activation of Akt.

To gain insight into in vivo functions of relevant genes in bladder cancer, an IPTG inducible expression system was designed where PTEN could be expressed in a PTEN deficient cell line (UMUC-3) (Herlevsen et al., 2007). These cells were injected subcutaneously in nude mice and IPTG was fed to these mice to induce PTEN expression, which did result in reduced tumor growth (p-value=0.01). Further use of this system allowed novel PTEN interacting proteins to be identified from the above cell line and analyzed by mass spectroscopy.

The present invention encompasses methods for generating a double knockout model animal having both p53 and Pten genes disrupted. In one embodiment, the method comprises using an adenovirus-Cre delivery approach to achieve sporadic deletion of tumor suppressor function specifically in the bladder urothelium. This method comprises providing a transgenic animal having one or both alleles of a p53 gene and one or both alleles of a Pten gene that have at least one LoxP site incorporated therein; and injecting an adenovirus-cre into one or more tissues of the animal, wherein the retroviral construct results in the disruption of the target genes by the Cre protein.

This approach allows the examination of the consequences of deletion of various tumor suppressor genes in this tissue. Using this technique it was discovered that specific deletion of both p53 and Pten leads to the development of invasive bladder tumors with 100% penetrance by 4 months, including distant metastases to the liver and other tissues, which are also sites for bladder cancer metastases in humans. The histological appearance of these tumors is remarkably similar to invasive human bladder cancer. In addition, the present invention provides tools and methods for identifying therapeutic agents, methods of observing the effects of treatment, and animal models for the development of bladder diseases, not limited to cancer.

The transgenic animal of the invention provides an animal model for cancer, in particular bladder cancer. In one embodiment, the invention relates to methods for generating a double knockout animal model for bladder disease, for example, bladder cancer. The method comprises the in situ delivery of adenoviur-Cre to the bladder uroepithelium and disruption in at least one bladder cell of at least one allele of both a p53 gene and a Pten gene.

In another embodiment, the model comprises a transgenic mouse whose genome contains a disruption in at least one allele of both endogenous p53 and Pten genes. In a preferred embodiment, the model comprises a transgenic mouse whose genome contains a homozygous disruption of both alleles of the endogenous p53 gene and Pten gene, wherein said animal develops one or more signs or symptoms of cancer or of bladder cancer. The transgenic mouse of the invention displays at least one sign or symptom associated with cancer of the bladder.

In another embodiment, the invention provides a method to screen for potential therapeutic agents for the treatment of cancer. A potential therapeutic agent is administered to a first transgenic animal whose genome comprises a disruption of both the endogenous p53 and Pten genes. The first transgenic animal is maintained for a time sufficient to permit the detection of a change in one or more signs or symptoms of cancer in the transgenic animal. A second transgenic animal having the same genetic background as the first transgenic animal and whose genome also comprises a disruption of both the endogenous p53 and Pten genes is maintained under the same conditions as the first animal but does not receive the potential therapeutic agent. The first and second animals are observed for a change in at least one sign or symptom associated with cancer. A therapeutic agent which prevents one or more signs or symptoms of cancer in the first transgenic animal when compared to the second transgenic animal will be a potential therapeutic agent for the treatment or prevention of cancer, for example bladder cancer.

In a preferred embodiment, the sign or symptom of cancer is the development of tumors in the bladder. In a preferred embodiment, detection of tumors in the bladder of the animal is performed by sacrificing the first and second animal after a time sufficient for the detection of at least one tumor of the bladder in the first and second animals has elapsed and observing the tissue of the bladder using techniques well known in the art. In another preferred embodiment, the transgenic animal is a mouse.

In another embodiment, a potential therapeutic agent is administered to a first transgenic animal whose genome comprises a disruption of both the endogenous p53 and Pten genes and to a second transgenic animal having a different genetic background from the first transgenic animal and whose genome also comprises a disruption of both the endogenous p53 and Pten genes and the effects of the therapeutic agent on the development of cancer is compared in the two animals. Many signs or symptoms associated with cancer, and in particular, bladder cancer are known in the art which may be used to screen for the development of cancer. In some embodiments, the animals do not have to be sacrificed to detect a sign or symptom associated with cancer. In certain embodiments the transgenic animal contains a homozygous disruption of both p53 and Pten genes.

It is contemplated that other methods of detecting the tumors or other signs or symptoms of cancer can be substituted for microscopic examination of tissue without departing from the scope of the invention. Non limiting examples may include withdrawing a body fluid from the first and second animal and analyzing the body fluid such as blood, for example, for the presence of one or more signs or symptoms of cancer of the bladder. By way of example, exfoliated cancer cells in urine or blood samples could be examined for reaction with antibody for small cancer antigens as known by those skilled in the art. In the context of In the invention, nucleic acids and/or proteins are manipulated according to well known molecular biology techniques. Detailed protocols for numerous such procedures are described in, e.g., in Ausubel et al. Current Protocols in Molecular Biology (supplemented through 2000) John Wiley & Sons, New York (“Ausubel”); Sambrook et al. Molecular Cloning-A Laboratory Manual (2nd Ed.), Vol. 1-3, Cold Spring Harbor Laboratory, Cold Spring Harbor, N.Y., 1989 (“Sambrook”), and Berger and Kimmel Guide to Molecular Cloning Techniques, Methods in Enzymology volume 152 Academic Press, Inc., San Diego, Calif. (“Berger”).

Genotyping. In addition to, or in conjunction with the correlation of expression profiles and clinical data, it is often desirable to correlate expression patterns with the subject's genotype at one or more genetic loci or to correlate both expression profiles and genetic loci data with clinical data. The selected loci can be, for example, chromosomal loci corresponding to one or more member of the candidate library, polymorphic alleles for marker loci, or alternative disease related loci (not contributing to the candidate library) known to be, or putatively associated with, a disease (or disease criterion). Indeed, it will be appreciated, that where a (polymorphic) allele at a locus is linked to a disease (or to a predisposition to a disease), the presence of the allele can itself be a disease criterion.

Numerous well known methods exist for evaluating the genotype of an individual, including southern analysis, restriction fragment length polymorphism (RFLP) analysis, polymerase chain reaction (PCR), amplification length polymorphism (AFLP) analysis, single stranded conformation polymorphism (SSCP) analysis, single nucleotide polymorphism (SNP) analysis (e.g., via PCR, Taqman or molecular beacons), among many other useful methods. Many such procedures are readily adaptable to high throughput and/or automated (or semi-automated) sample preparation and analysis methods. Most, can be performed on nucleic acid samples recovered via simple procedures from the same sample as yielded the material for expression profiling. Exemplary techniques are described in, e.g., Sambrook, and Ausubel, supra.

By “down-regulate” it is meant that the expression of the gene, or level of RNAs or equivalent RNAs encoding one or more proteins, or activity of one or more proteins, such as p53 and Pten genes, is reduced below that observed in the absence of the nucleic acid molecules of the invention. In one embodiment, inhibition or down-regulation with enzymatic nucleic acid molecule, for example, a siRNA, microRNA, ribozyme, or the like, preferably is below that level observed in the presence of an enzymatically inactive or attenuated molecule that is able to bind to the same site on the target RNA, but is unable to cleave that RNA. In another embodiment, inhibition or down-regulation with antisense oligonucleotides is preferably below that level observed in the presence of, for example, an oligonucleotide with scrambled sequence or with mismatches. In another embodiment, inhibition or down-regulation of p53 proteins, and Pten genes with the nucleic acid molecule of the instant invention is greater in the presence of the nucleic acid molecule than in its absence.

By “gene” it is meant a nucleic acid that encodes RNA, for example, nucleic acid sequences including but not limited to a segment encoding a polypeptide.

“Complementarity” refers to the ability of a nucleic acid to form hydrogen bond(s) with another RNA sequence by either traditional Watson-Crick or other non-traditional types.

By “RNA” is meant a molecule comprising at least one ribonucleotide residue. By “ribonucleotide” or “2′-OH” is meant a nucleotide with a hydroxyl group at the 2′ position of a D-ribo-furanose moiety.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, for example, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetyltidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N-6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra).

By “modified bases” in this aspect is meant nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

By “nucleic acid molecule” as used herein is meant a molecule having nucleotides. The nucleic acid can be single, double, or multiple stranded and can comprise modified or unmodified nucleotides or non-nucleotides or various mixtures and combinations thereof.

By “vectors” is meant any nucleic acid-based technique used to deliver a desired nucleic acid, for example, bacterial plasmid, viral nucleic acid, HAC, BAC, and the like.

As used in herein “cell” is used in its usual biological sense, and does not refer to an entire multicellular organism. The cell can, for example, be in vivo, in vitro or ex vivo, e.g., in cell culture, or present in a multicellular organism, including, e.g., birds, plants and mammals such as humans, cows, sheep, apes, monkeys, swine, rats, mice, dogs, and cats. The cell can be prokaryotic (e.g., bacterial cell) or eukaryotic (e.g., mammalian or plant cell).

Oligonucleotides (eg; antisense, GeneBlocs) are synthesized using protocols known in the art as described in Caruthers et al., 1992, Methods in Enzymology 211, 3 19, Thompson et al., International PCT Publication No. WO 99/54459, Wincott et al., 1995, Nucleic Acids Res. 23, 2677 2684, Wincott et al., 1997, Methods Mol. Bio., 74, 59, Brennan et al, 1998, Biotechnol Bioeng., 61, 33 45, and Brennan, U.S. Pat. No. 6,001,311. All of these references are incorporated herein by reference. In a non-limiting example, small scale syntheses are conducted on a 394 Applied Biosystems, Inc. synthesizer. Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention can be modified extensively to enhance stability by modification with nuclease resistant groups, for example, 2′-amino, 2′-C-allyl, 2′-fluoro, 2′-O-methyl, 2′-H (for a review see Usman and Cedergren, 1992, TIBS17, 34; Usman et al., 1994, Nucleic Acids Symp. Ser. 31, 163). While chemical modification of oligonucleotide internucleotide linkages with phosphorothioate, phosphorothioate, and/or 5′-methylphosphonate linkages improves stability, too many of these modifications can cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications that maintain or enhance activity are provided. Such nucleic acid is also generally more resistant to nucleases than unmodified nucleic acid. Nucleic acid molecules are preferably resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211, 3-19 (incorporated by reference herein) have expanded the ability to modify nucleic acid molecules by introducing nucleotide modifications to enhance their nuclease stability as described above.

Derivatives or analogs of the nucleic acids or proteins of the invention include, but are not limited to, molecules comprising regions that are substantially homologous to p53 or Pten nucleic acids by at least about 30%, 40%, 50%, 60%, 70%, 80%, 90%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins of the invention under stringent, moderately stringent, or low stringent conditions. See e.g. Ausubel, et al., CURRENT PROTOCOLS IN MOLECULAR BIOLOGY, John Wiley & Sons, New York, N.Y., 1993. Nucleic acid derivatives and modifications include those obtained by gene replacement, site-specific mutation, deletion, insertion, recombination, repair, shuffling, endonuclease digestion, PCR, subcloning, and related techniques.

“Homologs” can be naturally occurring, or created by artificial synthesis of one or more nucleic acids having related sequences, or by modification of one or more nucleic acids to produce related nucleic acids. Nucleic acids are homologous when they are derived, naturally or artificially, from a common ancestor sequence (e.g., orthologs or paralogs). If the homology between two nucleic acids is not expressly described, homology can be inferred by a nucleic acid comparison between two or more sequences. If the sequences demonstrate some degree of sequence similarity, for example, greater than about 30% at the primary amino acid structure level, it is concluded that they share a common ancestor. For purposes of the present invention, genes are homologous if the nucleic acid sequences are sufficiently similar to allow recombination and/or hybridization under low stringency conditions. As used herein “hybridization,” refers to the binding, duplexing, or hybridizing of a molecule only to a particular nucleotide sequence under low, medium, or highly stringent conditions, including when that sequence is present in a complex mixture (e.g., total cellular) DNA or RNA.

Furthermore, one of ordinary skill will recognize that mutations include the substitution, deletion or addition of polynucleotides or nucleic acids that alter, add or delete a single amino acid or a small number of amino acids in a coding sequence where the nucleic acid alterations result in the substitution of a chemically similar amino acid. Amino acids that may serve as conservative substitutions for each other include the following: Basic: Arginine (R), Lysine (K), Histidine (H); Acidic: Aspartic acid (D), Glutamic acid (E), Asparagine (N), Glutamine (Q); hydrophilic: Glycine (G), Alanine (A), Valine (V), Leucine (L), Isoleucine (I); Hydrophobic: Phenylalanine (F), Tyrosine (Y), Tryptophan (W); Sulfur-containing: Methionine (M), Cysteine (C). In addition, sequences that differ by conservative variations are generally homologous.

Descriptions of the molecular biological techniques useful to the practice of the invention including mutagenesis, PCR, cloning, and the like include Berger and Kimmel, GUIDE TO MOLECULAR CLONING TECHNIQUES, METHODS IN ENZYMOLOGY, volume 152, Academic Press, Inc., San Diego, Calif. (Berger); Sambrook et al., MOLECULAR CLONING—A LABORATORY MANUAL (2nd Ed.), Vol. 1-3, Cold Spring Harbor Laboratory, Cold Spring Harbor, N.Y., 1989, and CURRENT PROTOCOLS IN MOLECULAR BIOLOGY, F. M. Ausubel et al., eds., Current Protocols, a joint venture between Greene Publishing Associates, Inc. and John Wiley & Sons, Inc.; Berger, Sambrook, and Ausubel, as well as Mullis et al., U.S. Pat. No. 4,683,202 (1987); PCR PROTOCOLS A GUIDE TO METHODS AND APPLICATIONS (Innis et al. eds), Academic Press, Inc., San Diego, Calif. (1990) (Innis); Arnheim & Levinson (Oct. 1, 1990) C&EN 36-47. For suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

A polynucleotide can be a DNA molecule, a cDNA molecule, genomic DNA molecule, or an RNA molecule. A polynucleotide as DNA or RNA can include a sequence wherein T (thymidine) can also be U (uracil). If a nucleotide at a certain position of a polynucleotide is capable of forming a Watson-Crick pairing with a nucleotide at the same position in an anti-parallel DNA or RNA strand, then the polynucleotide and the DNA or RNA molecule are complementary to each other at that position. The polynucleotide and the DNA or RNA molecule are substantially complementary to each other when a sufficient number of corresponding positions in each molecule are occupied by nucleotides that can hybridize with each other in order to effect the desired process.

Transformation of a host cell with recombinant DNA may be carried out by conventional techniques as are well known to those skilled in the art. By “transformation” is meant a permanent or transient genetic change induced in a cell following incorporation of new DNA (i.e., DNA exogenous to the cell).

In any of the embodiments, the nucleic acids can be present as: one or more naked DNAs; one or more polynucleotides or nucleic acids disposed in an appropriate expression vector and maintained episomally; one or more polynucleotides or nucleic acids incorporated into the host cell's genome; a modified version of an endogenous gene encoding the components of the complex; one or more polynucleotides or nucleic acids in combination with one or more regulatory nucleic acid sequences; or combinations thereof. The nucleic acid may optionally comprise a linker peptide or fusion protein component, for example, His-Tag, FLAG-Tag, fluorescent protein, GST, TAT, an antibody portion, a signal peptide, and the like, at the 5′ end, the 3′ end, or at any location within the ORF.

When the host is a eukaryote, such methods of transfection with DNA include calcium phosphate co-precipitates, conventional mechanical procedures such as microinjection, electroporation, insertion of a plasmid encased in liposomes, or virus vectors, as well as others known in the art, may be used. The eukaryotic cell may be a yeast cell (e.g., Saccharomyces cerevisiae) or may be a mammalian cell, including a human cell.

By “antisense nucleic acid”, it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop or hairpin, and/or an antisense molecule can bind such that the antisense molecule forms a loop or hairpin. Thus, the antisense molecule can be complementary to two (or even more) non-contiguous substrate sequences or two (or even more) non-contiguous sequence portions of an antisense molecule can be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol, 40, 1-49, which are incorporated herein by reference in their entirety. In addition, antisense DNA can be used to target RNA by means of DNA-RNA interactions, thereby activating RNase H, which digests the target RNA in the duplex. The antisense oligonucleotides can comprise one or more RNAse H activating region, which is capable of activating RNAse H cleavage of a target RNA. Antisense DNA can be synthesized chemically or expressed via the use of a single stranded DNA expression vector or equivalent thereof.

By “enzymatic nucleic acid molecule” it is meant a nucleic acid molecule which has complementarity in a substrate binding region to a specified gene target, and also has or mediates an enzymatic activity which is active to specifically cleave target RNA. That is, the enzymatic nucleic acid molecule is able to intermolecularly cleave RNA, alone or as a component of an enzymatic complex, and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% can also be useful in this invention (see for example Werner and Uhlenbeck, 1995, Nucleic Acids Research, 23, 2092 2096; Hammann et al., 1999, Antisense and Nucleic Acid Drug Dev., 9, 25 31). The nucleic acids can be modified at the base, sugar, and/or phosphate groups. The term “enzymatic nucleic acid” is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, siRNA, micro RNA, short hairpin RNA, endoribonuclease, RNA-induced silencing complexes, endonuclease, minizyme, leadzyme, oligozyme or DNA enzyme. All of these terminologies describe nucleic acid molecules with enzymatic activity.

The specific enzymatic nucleic acid molecules described in the instant application are not limiting in the invention and those skilled in the art will recognize that all that is important in an enzymatic nucleic acid molecule of this invention is that it has a specific substrate binding site which is complementary to one or more of the target nucleic acid regions, and that it have nucleotide sequences within or surrounding that substrate binding site which impart a nucleic acid cleaving and/or ligation activity to the molecule (Cech et al., U.S. Pat. No. 4,987,071; Cech et al., 1988, 260 JAMA 3030).

Several varieties of enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Thus, a single ribozyme molecule is able to cleave many molecules of target RNA. In addition, the ribozyme is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of a ribozyme.

By “equivalent” or “related” RNA to p53 or Pten genes is meant to include those naturally occurring -RNA molecules having homology (partial or complete) to p53 or Pten proteins, p53 or Pten binding proteins, and genes encoding for proteins with similar function as p53 or Pten proteins, p53 or Pten binding proteins in various organisms, including human, rodent, primate, rabbit, pig, protozoans, fungi, plants, and other microorganisms and parasites. The equivalent RNA sequence also includes in addition to the coding region, regions such as 5′-untranslated region, 3′-untranslated region, introns, intron-exon junction and the like. By “homology” is meant the nucleotide sequence of two or more nucleic acid molecules is partially or completely identical. In certain embodiments the homolgous nucleic acid has 30%, 40%, 50%, 60%, 70%, 80%, 90%, or 95% homology to p53 or Pten, or p53 or Pten binding proteins.

Long double-stranded RNAs (dsRNAs; typically >200 nt) can be used to silence the expression of target genes in a variety of organisms and cell types (e.g., worms, fruit flies, and plants). Upon introduction, the long dsRNAs enter a cellular pathway that is commonly referred to as the RNA interference (RNAi) pathway. First, the dsRNAs get processed into 20-25 nucleotide (nt) small interfering RNAs (siRNAs) by an RNase III-like enzyme called Dicer (initiation step). Then, the siRNAs assemble into endoribonuclease-containing complexes known as RNA-induced silencing complexes (RISCs), unwinding in the process. The siRNA strands subsequently guide the RISCs to complementary RNA molecules, where they cleave and destroy the cognate RNA (effecter step). Cleavage of cognate RNA takes place near the middle of the region bound by the siRNA strand. In mammalian cells, introduction of long dsRNA (>30 nt) initiates a potent antiviral response, exemplified by nonspecific inhibition of protein synthesis and RNA degradation. The mammalian antiviral response can be bypassed, however, by the introduction or expression of siRNAs.

Injection and transfection of dsRNA into cells and organisms has been the main method of delivery of siRNA. And while the silencing effect lasts for several days and does appear to be transferred to daughter cells, it does eventually diminish. Recently, however, a number of groups have developed expression vectors to continually express siRNAs in transiently and stably transfected mammalian cells. (See, e.g., Brummelkamp T R, Bemards R, and Agami R. (2002). A system for stable expression of short interfering RNAs in mammalian cells. Science 296:550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9):6047-6052, which are herein incorporated by reference in their entirety).

Some vectors have been engineered to express small hairpin RNAs (shRNAs), which get processed in vivo into siRNAs-like molecules capable of carrying out gene-specific silencing. The vectors contain the shRNA sequence between a polymerase III (pol III) promoter (e.g., U6 or H1 promoters) and a 4-5 thymidine transcription termination site. The transcript is terminated at position 2 of the termination site (pol III transcripts naturally lack poly(A) tails) and then folds into a stem-loop structure with 3′ UU-overhangs. The ends of the shRNAs are processed in vivo, converting the shRNAs into ˜21 nt siRNA-like molecules, which in turn initiate RNAi. This latter finding correlates with recent experiments in C. elegans, Drosophila, plants and Trypanosomes, where RNAi has been induced by an RNA molecule that folds into a stem-loop structure.

In another aspect of the invention, enzymatic nucleic acid molecules or antisense molecules that interact with target RNA molecules, and down-regulate p53 or Pten genes, or p53 or Pten binding protein genes are expressed from transcription units inserted into DNA or RNA vectors. The recombinant vectors are preferably DNA plasmids or viral vectors. The use of RNAi vectors and expression systems is known and are commercially available from Ambion, Inc.® (Austin, Tex.), Lentigen, Inc. (Baltimore, Md.), Panomics (Fremont, Calif.), Open Biosystems (e.g., Expression Arrest™; Huntsvill, Ala.), and Sigma-Aldrich (ST. Louis, Mo.).

Enzymatic nucleic acid molecule or antisense expressing viral vectors can be constructed based on, but not limited to, lenti virus, cytomegalovirus, adeno-associated virus, retrovirus, adenovirus, or alphavirus. Preferably, the recombinant vectors capable of expressing the enzymatic nucleic acid molecules or antisense are delivered, and persist in target cells. Alternatively, viral vectors can be used that provide for expression of enzymatic nucleic acid molecules or antisense. Such vectors can be repeatedly administered as necessary. Once expressed, the enzymatic nucleic acid molecules or antisense bind to the target RNA and down-regulate its function or expression. Delivery of enzymatic nucleic acid molecule or antisense expressing vectors can be systemic, such as by intravenous or intramuscular administration, by administration to target cells explanted from the patient or subject followed by reintroduction into the patient or subject, or by any other means that would allow for introduction into the desired target cell. Antisense DNA can be expressed via the use of a single stranded DNA intracellular expression vector.

The nucleic acid molecules of the instant invention, individually, or in combination or in conjunction with other drugs, can be used to modify the expression of p53 or Pten genes.

The use of specially designed vector constructs for inducing RNA interference has numerous advantages over oligonucleotide-based techniques. The most significant advantages are stability, and induced transcription via inducible promoters. Promoter regions in the vector ensure that shRNA transcripts are constantly expressed, maintaining cellular levels at all times. Thus, the duration of the effect is not as transient as with injected RNA, which usually lasts no longer than a few days. And by using expression constructs instead of oligo injection, it is possible to perform multi-generational studies of gene knockdown because the vector can become a permanent fixture in the cell line.

By “triplex forming oligonucleotides” or “triplex oligonucleotide” is meant an oligonucleotide that can bind to a double-stranded DNA in a sequence-specific manner to form a triple-strand helix. Formation of such triple helix structure has been shown to inhibit transcription of the targeted gene (Duval-Valentin et al., 1992 Proc. Natl. Acad. Sci. USA 89, 504; Fox, 2000, Curr. Med. Chem., 7, 17-37; Praseuth et. al., 2000, Biochim. Biophys. Acta, 1489, 181-206).

By “double stranded RNA” or “dsRNA” is meant a double stranded RNA that matches a predetermined gene sequence that is capable of activating cellular enzymes that degrade the corresponding messenger RNA transcripts of the gene. These dsRNAs are referred to as short intervening RNA (siRNA) and can be used to inhibit gene expression (see for example Elbashir et al., 2001, Nature, 411, 494-498; and Bass, 2001, Nature, 411, 428-429). The term “double stranded RNA” or “dsRNA” as used herein refers to a double stranded RNA molecule capable of RNA interference “RNAi”, including short interfering RNA “siRNA” see for example Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914. The use of inhibitory RNA molecules and techniques are known in the art and are described in detail in U.S. Pat. No. 7,022,828, the teachings of which are incorporated herein by reference in their entirety for all purposes.

In one embodiment of the present invention, a nucleic acid molecule of the instant invention can be between about 10 and 100 nucleotides in length. For example, RNAi nucleic acid molecules of the invention are preferably between about 15 and 50 nucleotides in length, more preferably between about 25 and 40 nucleotides in length (for example see Jarvis et al., 1996, J. Biol. Chem., 271, 29107 29112). Exemplary inhibitory RNA molecules of the invention are preferably between about 15 and 75 nucleotides in length, more preferably between about 20 and 35 nucleotides in length (see for example Woolf et al., 1992, PNAS., 89, 7305 7309; Milner et al., 1997, Nature Biotechnology, 15, 537 541). Exemplary triplex forming oligonucleotide molecules of the invention are preferably between about 10 and 40 nucleotides in length, more preferably between about 12 and 25 nucleotides in length (see for example Maher et al, 1990, Biochemistry, 29, 8820 8826; Strobel and Dervan, 1990, Science, 249, 73 75). Those skilled in the art will recognize that all that is required is that the nucleic acid molecule be of sufficient length and suitable conformation for the nucleic acid molecule to interact with its target and/or catalyze a reaction contemplated herein. The length of the nucleic acid molecules of the instant invention are not limiting within the general limits stated. Preferably, a nucleic acid molecule that modulates, for example, down-regulates p53 or Pten, or p53 or Pten binding protein gene expression comprises between 12 and 100 bases complementary to a RNA molecule of a 53 or Pten gene, a 53 or Pten binding protein gene.

The invention provides a method for producing a class of nucleic acid-based gene modulating agents which exhibit a high degree of specificity for the RNA of a desired target. For example, the enzymatic nucleic acid molecule is preferably targeted to a highly conserved sequence region of target RNAs encoding a 53 or Pten, 53 or Pten binding protein gene such that specific treatment can be provided with either one or several nucleic acid molecules of the invention. Such nucleic acid molecules can be delivered exogenously to specific tissue or cellular targets as required. Alternatively, the nucleic acid molecules (e.g., ribozymes and antisense) can be expressed from DNA and/or RNA vectors that are delivered to specific cells.

The nucleic acid-based inhibitors of the invention are added directly, or can be complexed with cationic lipids, packaged within liposomes, or otherwise delivered to target cells or tissues. The nucleic acid or nucleic acid complexes can be locally administered to relevant tissues in vitro, ex vivo, or in vivo through injection or infusion pump, with or without their incorporation in biopolymers.

In another embodiment, the invention features an enzymatic nucleic acid molecule having one or more non-nucleotide moieties, and having enzymatic activity to cleave an RNA or DNA molecule.

In a further embodiment, the described nucleic acid molecules, such as antisense or ribozymes, can be used in combination with other known treatments to treat conditions or diseases discussed above. For example, the described molecules can be used in combination with one or more known therapeutic agents.

RNAi molecules can be modified or unmodified RNA, DNA, or mixed polymer oligonucleotides and primarily function by specifically binding to matching sequences resulting in inhibition of peptide synthesis (Wu-Pong, November 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. RNAi molecules can also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA can result in nuclease degradation of the heteroduplex (Wu-Pong, supra; Crooke, supra). To date, the only backbone modified DNA chemistry which acts as substrates for RNase H are phosphorothioates, phosphorodithioates, and borontrifluoridates. Recently it has been reported that 2′-arabino and 2′-fluoro-arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., International PCT Publication No. WO 99/54459; Hartmann et al., U.S. Ser. No. 60/101,174 which was filed on Sep. 21, 1998) all of these are incorporated by reference herein in their entirety.

Several varieties of enzymatic RNAs are presently known. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83 87; Beaudry et al., 1992, Science 257, 635-641; Joyce, 1992, Scientific American 267, 90-97; Breaker et al., 1994, TIBTECH 12, 268; Bartel et al., 1993, Science 261:1411-1418; Szostak, 1993, TIBS17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakacane & Eckstein, 1994, supra; Long & Uhlenbeck, 1994, supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions.

The enzymatic nature of an enzymatic nucleic acid molecule can allow the concentration of enzymatic nucleic acid molecule necessary to affect a therapeutic treatment to be lower. This reflects the ability of the enzymatic nucleic acid molecule to act enzymatically. Thus, a single enzymatic nucleic acid molecule is able to cleave many molecules of target RNA. In addition, the enzymatic nucleic acid molecule is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to greatly attenuate the catalytic activity of a enzymatic nucleic acid molecule.

Nucleic acid molecules having an endonuclease enzymatic activity are able to repeatedly cleave other separate RNA molecules in a nucleotide base sequence-specific manner. Such enzymatic nucleic acid molecules can be targeted to virtually any RNA transcript, and achieve efficient cleavage in vitro (Zaug et al., 324, Nature 429 1986; Uhlenbeck, 1987 Nature 328, 596; Kim et al., 84 Proc. Natl. Acad. Sci. USA 8788, 1987; Dreyfus, 1988, Einstein Quart. J. Bio. Med., 6, 92; Haseloff and Gerlach, 334 Nature 585, 1988; Cech, 260 JAMA 3030, 1988; and Jefferies et al., 17 Nucleic Acids Research 1371, 1989; Santoro et al., 1997 supra).

Because of their sequence specificity, trans-cleaving enzymatic nucleic acid molecules can be used as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional and abrogates protein expression from that RNA. In this manner, synthesis of a protein associated with a disease state can be selectively inhibited (Warashina et al., 1999, Chemistry and Biology, 6, 237-250).

Enzymatic nucleic acid molecules of the invention that are allosterically regulated (“allozymes”) can be used to modulate 53 or Pten, or 53 or Pten binding protein gene expression. These allosteric enzymatic nucleic acids or allozymes (see for example George et al, U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842) are designed to respond to a signaling agent, which in turn modulates the activity of the enzymatic nucleic acid molecule and modulates expression of 53 or Pten, or 53 or Pten binding protein gene. In response to interaction with a predetermined signaling agent, the allosteric enzymatic nucleic acid molecule's activity is activated or inhibited such that the expression of a particular target is selectively down-regulated.

Oligonucleotides (eg; antisense, GeneBlocs) are synthesized using protocols known in the art as described in Caruthers et al., 1992, Methods in Enzymology 211, 3 19, Thompson et al., International PCT Publication No. WO 99/54459, Wincott et al., 1995, Nucleic Acids Res. 23, 2677 2684, Wincott et al., 1997, Methods Mol. Bio., 74, 59, Brennan et al, 1998, Biotechnol Bioeng., 61, 33 45, and Brennan, U.S. Pat. No. 6,001,311. All of these references are incorporated herein by reference. In a non-limiting example, small scale syntheses are conducted on a 394 Applied Biosystems, Inc. synthesizer. Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

In one embodiment, the invention features modified enzymatic nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331 417, and Mesmaeker et al., 1994, Novel Backbone Replacements for Oligonucleotides, in Carbohydrate Modifications in Antisense Research, ACS, 24 39. These references are hereby incorporated by reference herein. Various modifications to nucleic acid (e.g., antisense and ribozyme) structure can be made to enhance the utility of these molecules. For example, such modifications can enhance shelf-life, half-life in vitro, bioavailability, stability, and ease of introduction of such oligonucleotides to the target site, including e.g., enhancing penetration of cellular membranes and conferring the ability to recognize and bind to targeted cells.

Administration of Nucleic Acid Molecules. Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995 which are both incorporated herein by reference. Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by a incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to oral (tablet or pill form) and/or intrathecal delivery (Gold, 1997, Neuroscience, 76, 1153-1158). Other approaches include the use of various transport and carrier systems, for example, through the use of conjugates and biodegradable polymers. For a comprehensive review on drug delivery strategies including CNS delivery, see Ho et al., 1999, Curr. Opin. Mol. Ther., 1, 336-343 and Jain, Drug Delivery Systems: Technologies and Commercial Opportunities, Decision Resources, 1998 and Groothuis et al., 1997, J. NeuroVirol., 3, 387-400.

ELISA Assay. An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., Fab or F(ab)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Thory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques intracavity, or transdermally, alone or with effector cells.

A transgenic cell or animal of the invention can include a transgene. The transgene can disrupt or encode a protein that is normally exogenous to the transgenic cell or animal, including a human protein. The transgene can be linked to a heterologous or a native promoter.

This disclosure further relates to a method of producing transgenic animals. Techniques known in the art may be used to introduce the transgene into animals to produce the founder line of animals. Such techniques include, but are not limited to: pronuclear microinjection; retrovirus mediated gene transfer into germ lines (Van der Putten et al., Proc. Natl. Acad. Sci. USA 82: 6148-6152, 1985; gene targeting in embryonic stem cells (Thompson et al., Cell 56: 313-321, 1989; electroporation of embryos (Lo, Mol. Cell. Biol. 3: 1803-1814, 1983; and sperm-mediated gene transfer (Lavitrano, et al., Cell 57: 717-723, 1989; etc. For a review of such techniques, see Gordon, Intl. Rev. Cytol. 115: 171-229, 1989. Accordingly, the invention features a transgenic organism that contains a transgene encoding a chimeric interleukin/interleukin receptor polypeptide. The transgenic organism can be a eukaryotic cell, for example, a yeast cell, an insect, e.g., a worm or a fly, a fish, a reptile, a bird, or a mammal, e.g., a rodent such as a mouse. The transgenic organism can further comprise a genetic alteration, e.g., a point mutation, insertion, or deficiency, in an endogenous gene.

The invention further provides methods for producing host cells of the invention. In one embodiment, the method comprises culturing the host cell of invention (into which an exogenous nucleic acid has been introduced). In another embodiment, the method further comprises isolating a host cell from a transgenic animal. The term “host cell” includes a cell that might be used to carry a heterologous nucleic acid or has a disruption of an endogenous gene. A host cell can contain genes that are not found within the native (non-recombinant) form of the cell, genes found in the native form of the cell where the genes are modified and re-introduced into the cell by artificial means, or a nucleic acid endogenous to the cell that has been artificially modified without removing the nucleic acid from the cell. A host cell may be eukaryotic or prokaryotic. General growth conditions necessary for the culture of bacteria can be found in texts such as BERGEY'S MANUAL OF SYSTEMATIC BACTERIOLOGY, Vol. 1, N. R. Krieg, ed., Williams and Wilkins, Baltimore/London (1984). A “host cell” can also be one in which the endogenous genes or promoters or both have been modified.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding the protein or can be introduced on a separate vector. Cells stably transfected with the introduced nucleic acid can be identified by drug selection (e.g., cells that have incorporated the selectable marker gene will survive, while the other cells die).

The host cells of the invention can also be used to produce non-human transgenic animals. For example, in one embodiment, a host cell of the invention is a fertilized oocyte or an embryonic stem cell into which the genetic disruption has been introduced. Such host cells can then be used to create non-human transgenic animals in which exogenous sequences have been introduced into their genome or homologous recombinant animals in which endogenous nucleic acid sequences have been altered. Such animals are useful for studying the function and/or activity of proteins and for identifying and/or evaluating modulators of protein activity. As used herein, a “transgenic animal” is a non-human animal, preferably a mammal, more preferably a rodent such as a rat or mouse, in which one or more of the cells of the animal includes a genetic disruption or a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

The invention provides a transgenic animal whose genome comprises a homozygous disruption of both the endogenous p53 gene (e.g., SEQ ID NO:1) and Pten (e.g., SEQ ID NO:2) genes wherein the animal's genome can additionally comprise a DNA sequence encoding a heterologous gene of interest. The gene of interest may code for a biologically active nucleic acid or polypeptide including, for example, an an immunomodulator, a peptide, an oligonucleotide and the like. The gene of interest can also be inserted within a target gene of the transgenic animal of the invention in order to disrupt that target gene, thereby generating a knockout mouse having a homozygous disruption of the p53, Pten target genes. The heterologous gene of interest can comprise, for example, an antibiotic marker gene or an allelic variant of the gene to be disrupted, wherein the allelic variant is not expressed or is not biologically active. In addition, genes can be disrupted by providing an antisense RNA, a ribozyme and the like to prevent transcription or translation of the target gene.

The invention further provides a method for assessing the therapeutic effect of a heterologous gene of interest on the development of bladder cancer which comprises expressing the heterologous gene of interest in a first transgenic animal whose genome comprises a homozygous disruption of both the endogenous p53 gene and Pten gene. The first transgenic animal is maintained for a time sufficient to permit the detection a change in one or more signs or symptoms in the first transgenic animal associated with bladder cancer. A second transgenic animal having the same genetic background as the first transgenic animal and comprising a homozygous disruption of both the endogenous p53 gene and Pten gene which does not express the gene of interest is maintained under the same conditions as the first transgenic animal. Both transgenic animals are observed for the presence or absence of one or more signs or symptoms of bladder cancer.

In another embodiment, a heterologous gene of interest can be expressed in a first transgenic animal whose genome comprises a homozygous disruption of both the endogenous p53 and Pten genes. The heterologous gene of interest is not expressed in a second animal having a homozygous disruption in both the endogenous p53 and Pten genes but the first and second animals have different genetic backgrounds.

The invention further provides a method of identifying markers associated with bladder disease, or bladder cancer, the method comprising comparing the presence, absence or level of expression of at least one gene or protein in a transgenic animal whose genome comprises a homozygous disruption of both the endogenous p53 and Pten genes with the level or expression of the gene or protein in a second animal, wherein the second animal has the same genetic background as the first animal but does not comprise a homozygous disruption of both the endogenous p53 and Pten genes, wherein the difference between the first transgenic animal and the second animal in the presence, absence or level of expression of the gene or protein indicates that the expression of the gene is a marker associated with cancer of the bladder.

The invention further provides cells isolated from the knockout mice of the invention. Such cells can be of any cell type that can be isolated from the transgenic animal, utilizing techniques well known in the art. By way of example, isolated cells can include stem cells, uroepithelial cells, myofibroblasts and the like. The cells can be utilized in in vitro experiments to study the physiologic characteristics of such cells and can comprise cell lines from the knockout mice.

General Methods

The practice of the present invention employs, unless otherwise indicated, conventional techniques of chemistry, molecular biology, microbiology, recombinant DNA, genetics, immunology, cell biology, cell culture and transgenic biology, which are known to one of ordinary skill in the related art.

Genotyping of p53 and Pten mice was done with either Wester, Southern or PCR analysis of DNA isolated from bladders. Transgenic animals having a heterozygous or homozygous disruption in one or more genes can also be crossed to other animals having the same or different homozygous or heterozygous disruptions in the same or different genes to generate numerous combinations of heterozygous and homozygous disruptions of multiple genes, as well known in the art and as demonstrated in the Examples of the present invention.

Furthermore, a transgenic animal of the invention can be transformed with a heterologous gene of interest having a disruption in order to modulate the expression of the heterologous gene in an animal having a homozygous disruption of the P53 and Pten genes. In this manner, one can determine the effects of modulating the expression of a heterologous gene of interest on a transgenic animal having a homozygous disruption of the P53 and Pten genes.

As used herein, the term “heterologous gene” or “heterologous nucleic acid sequence” refers to a sequence that originates from a foreign species, or, if from the same species, it may be substantially modified from its original form. The term also encompasses an unchanged nucleic acid sequence that is not normally expressed in a cell. Preferably, the heterologous sequence is operably linked to a promoter, resulting in a chimeric gene. In preferred embodiments, the heterologous gene of interest is associated with either an increase or decrease in at least one sign or symptom of bladder disease or bladder cancer. It may also be desirable to observe the effect of a biological response modifier incorporated into the genome of the transgenic animal of the invention. Included in this category are immunopotentiating agents including nucleic acids encoding a number of the cytokines classified as interleukins, interferons, tumor necrosis factor (TNF) tumor suppressor genes, anti-angiogenic genes and the like. See, for example, U.S. Pat. Nos. 6,288,024; 4,879,226; and 6,300,475.

The terms “knockout” and “disruption” each refer to partial or complete reduction of the expression of at least a portion of a nucleic acid or a polypeptide encoded by one or more endogenous genes of a single cell, selected cells, or all of the cells of an animal. The animal may be a “heterozygous knockout” or have a “heterozygous disruption,” wherein one allele of one or more endogenous genes have been disrupted. Alternatively, the animal may be a “homozygous knockout” or have a “homozygous disruption,” wherein both alleles of one or more endogenous genes have been disrupted.

Methods of generating transgenic mice by inserting a nucleic acid sequence which can cause a disruption in an endogenous gene into the pronuclei of a fertilized mouse oocyte are known in the art (See e.g., U.S. Pat. No. 4,736,866 issued to Leder, et al.). Typically, the sequence is inserted into an undifferentiated cell termed an embryonic stem cell (ES cell). ES cells are usually derived from an embryo or blastocyst of the same species as the developing embryo into which it can be introduced. The knockout sequence can cause a disruption in a gene by insertion of an altered nucleic acid sequence into a homologous region of the coding region of the endogenous nucleic acid sequence (usually containing one or more exons) and/or the promoter region of a gene so as to decrease or prevent expression of the full length gene product in the cell. Insertion is usually accomplished by homologous recombination. Such methods are known in the art. By way of example, a disruption construct can be prepared by inserting, for example, a nucleotide sequence comprising an antibiotic resistance gene into a portion of an isolated nucleotide sequence encoding an endogenous gene that is to be disrupted. When this knockout construct is then inserted into an embryonic stem cell, the construct can integrate into the genomic DNA of at least one allele of the gene. Thus, many progeny of the cell will have the gene disrupted and no longer express the nucleic acid or gene or will express it at a decreased level and/or in a truncated form. Also, use of oligonucleotides or antisense nucleic acids which are complementary to at least a portion of a specific mRNA molecule to stall transcription of the mRNA can also be utilized to disrupt gene expression.

Examples. It will be understood that the embodiments and examples described herein are given by way of examples and are not intended to be limiting on the scope of the present invention.

Cre-Lox recombination involves the targeting of a specific sequence of DNA and splicing it with the help of an enzyme called Cre recombinase. Using this technology, specific tissue types or cells of organisms can be genetically modified, whilst other tissue remains unchanged. The Cre/lox system is used as a genetic tool to control site specific recombination events in genomic DNA. This system has allowed researchers to manipulate a variety of genetically modified organisms to control gene expression, delete undesired DNA sequences and modify chromosome architecture. The system begins with the Cre protein, a site-specific DNA recombinase. Cre can catalyse the recombination of DNA between specific sites in a DNA molecule. These sites, known as loxP sequences, contain specific binding sites for Cre that surround a directional core sequence where recombination can occur.

When cells that have loxP sites in their genome express Cre, a reciprocal recombination event will occur between the loxP sites. The double stranded DNA is cut at both loxP sites by the Cre protein and then ligated (glued) back together. It is a quick and efficient process. The efficiency of recombination depends on the orientation of the loxP sites. For two lox sites on the same chromosome arm, inverted loxP sites will cause an inversion, while a direct repeat of loxP sites will cause a deletion event. If loxP sites are on different chromosomes it is possible for translocation events to be catalysed by Cre induced recombination.

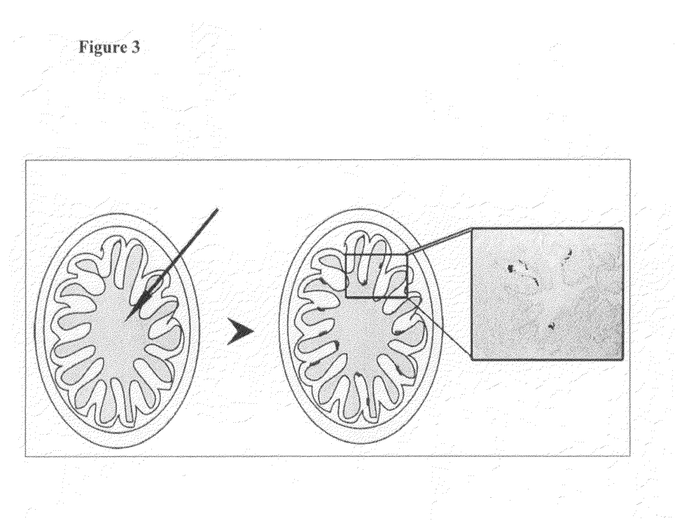

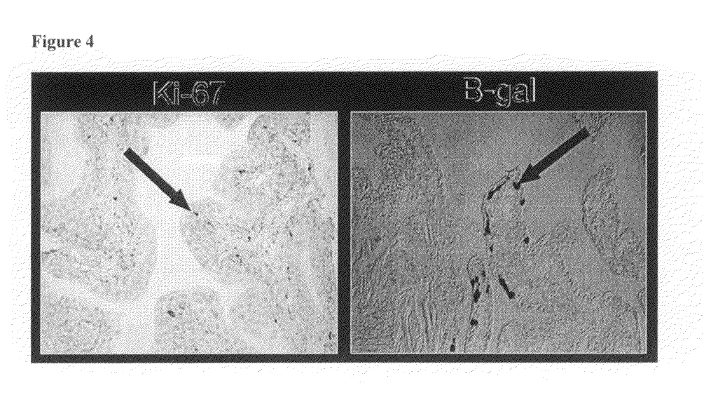

Generation of a mouse model of invasive bladder cancer. Invasive bladder cancer is thought to arise from somatic mutations that occur randomly in the uroepithelium cell layer. To try and model a similar initiation of bladder cancer in the mouse, a R26r reporter mouse was used and ˜2×10⁸ pfu of Adenovirus-cre was injected through the bladder muscle layer directly into the lumen. It was reasoned that since the uroepithelial cells are facing the lumen that they would be preferentially targeted for infection by the Adenovirus. It was found that ˜2×10⁸ pfu of Adenovirus-cre allowed for random infection of approximately 10% of uroepithelial cells, specifically, and using R26r mice allowed for visualization of these targeted cells using β-galactosidase staining (FIG. 3). Adenovirus preferable infects cells that are proliferating and interestingly the cell layer that is Ki-67 positive and proliferating contains the same cells that are β-galactosidase positive (FIG. 4). For this study, mostly adult male mice were used that were of approximately 2-3 months of age, though females also developed tumors and current investigation of injection into older mice is ongoing.

Certain genetic alterations have been proposed to be more common in invasive bladder cancer. To understand these alterations and the pathways that are affected in bladder cancer, different conditional mice that had either floxed tumor suppressor genes that allow for conditional gene knock-out or floxed-stop-floxed oncogenes that allow for gene activation were crossed in combinations that are relevant for bladder cancer or that are known to be important because of their involvement in other cancers. Table 1 summarizes all the genetic combinations of mice generated and injected with Adenovirus-cre into the bladder lumen and details of findings. No control (R26r^{R26r/R26r}) or single mutant mice (p53^{LoxP/LoxP}, Pten^{LoxP/LoxP}, Rb^{LoxP/LoxP}) injected showed signs of tumors even up to 12 months of age.

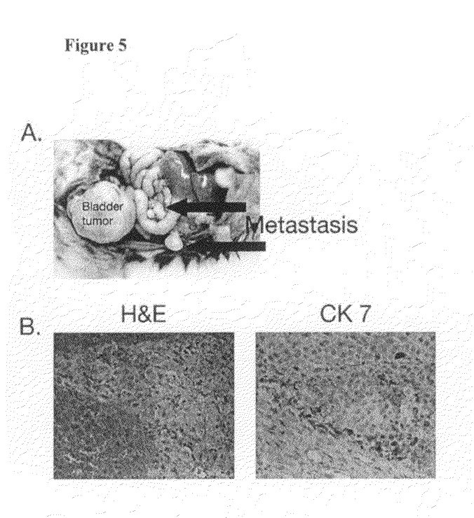

Suprisingly, p53^{LoxP/LoxP}; Rb^{LoxP/LoxP} mice which were monitored up to 12 months also did not develop tumors even though human data has shown their importance in invasive bladder cancer progression. In contrast, p53^{LoxP/LoxP}-Pten^{LoxP/LoxP} mice began to develop tumors as early as 2 months after adenovirus injection and up to 5 months (mice succumbed to the disease from 2.8 to 6 months). Addition of an activatable Kras (Kras^{LoxP/+}; p53^{LoxP/+}; Pten^{LoxP/LoxP} and Kras^{LoxP/+}; p53^{LoxP/LoxP}; Pten^{LoxP/LoxP}), resulted in acceleration of muscle invasive bladder cancer and death even though the tumors on average in these mice were smaller (423.96±73.058 and 112.35±21.5, respectably) than in p53^{LoxP/LoxP} Pten^{LoxP/LoxP} mice (for example, at 2.5-4.0 mos 709.55±78.839) and lacked visible metastases in a majority of the animals (Table 1, FIG. 5).

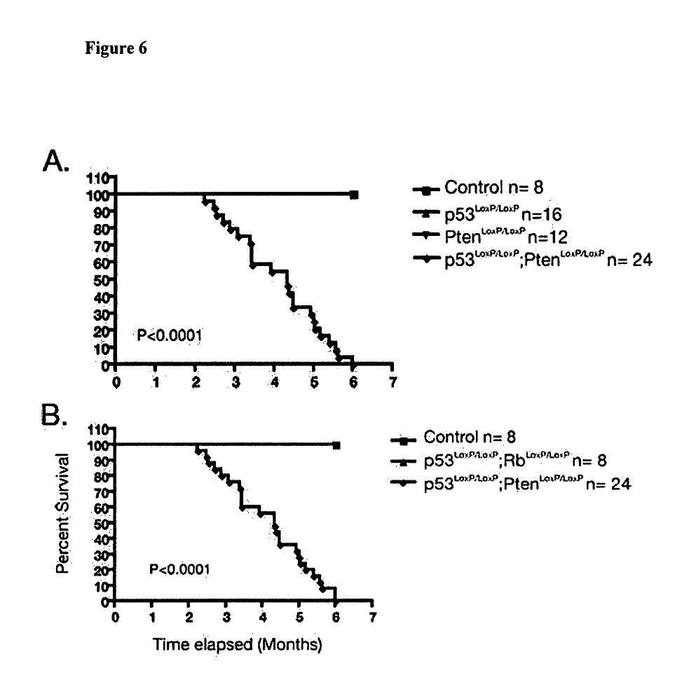

Survival curves were calculated by examining mice by palpitation for bladder tumors on a regular basis and dissecting any animal whose tumor was determined to large for survival. Calculating the survival curves of the control, single mutant (p53^{LoxP/Loxp}, Pten^{LoxP/LoxP}), and double mutant mice (p53^{LoxP/LoxP}; Pten^{LoxP/LoxP}) up to 6 months after injection revealed that even though all mice were injected with the same adenovirus and kept under the same conditions no control or single mutant mice died (FIG. 6). In sharp contrast, the survival of p53^{LoxP/LoxP}; Pten^{LoxP/LoxP} mice began to decrease at 2.5 months. At X months, the percent survival was already 50% and all mice succumbed to bladder cancer by 6 months after injection which was highly reproducible (p<0.0001).

TABLE 1
Genotypes of mice analyzed for bladder cancer with information on time of dissection after Adenovirus
injection, number of mice analyzed, weights of normal bladders or tumors, occurrence of metastases
and other relevant comments. (data in parenthesis indicates not dissected).

| | Time | | Weights of | | |
|---|---|---|---|---|---|
| | after | | bladder/tumors | | |
| | injection | | (mgs) ± standard | Metastasis | |
| Genotype | (months) | N# | error | (visible) | Comments |
| Control (R26r) | 0.10-11.0 | 15 | 36.14 ± 0.334 | none | Normal histology, no tumors |
| | | | n# = 12 | | |
| p53 | 1.90-13.7 | 24 | 40.64 ± 0.643 | none | Infiltration of blood cells in |
| | | | n# = 18 | | some samples, no tumors |
| Pten | 1.1713.50 | 26 | 43.80 ± 1.990 | none | Inflammation, “big cell” |
| | | | n# = 22 | | phenotype, no tumors |
| Kras | (1.40) | (1) | n/a | n/a | n/a |
| KrasL/+; PtenL/+ | 3.43-8.30 | 12 | 39.61 ± 1.129 | none | Inflammation, no tumors |
| | | | n# = 11 | | |
| KrasL/+; PtenL/L | 6.0010.00 | 5 | 39.93 ± 0.910 | none | No tumors |
| KrasL/+; p53L/+ | (4.80-6.8) | (2) | n/a | n/a | n/a |
| KrasL/+; p53L/L | 2.87-5.00 | 4 | 40.95 ± 7.530 | none | |
| | | | n# = 2 | | |
| p53L/+; PtenL/+ | (2.00) | (3) | n/a | n/a | n/a |
| p53L/+; PtenL/L | 2.03-4.80 | 3 | 33.2 ± 0.000 | none | Proliferation, no tumors |
| | | | n# = 2 | | |
| p53L/L; PtenL/+ | 6.07 | 1 | 44.6 n# = 1 | none | No tumors |
| p53L/L; PtenL/L | 0.70-6.00 | 29 | 0.0-2.5 mos | None 0.0-2.5 mos, | Inflammation, multi-layered |
| | | | 151.35 ± 43.017 | 2/8 2.5-4.0 mos, | epithelial cells, example of early |
| | | | n# = 6, 2.5-4.0 mos | 5/10 | invasion, epithelial nests, |
| | | | 709.55 ± 78.839 | 4.0-6.0 mos | increased cytoplasmic:nuclear |
| | | | n# = 9, 4.0-6.0 mos | | ratio muscle invasiveness |
| | | | 2403.43 ± 240.343 | | |
| | | | n# = 10 | | |
| p53L/L; RbL/L | 3.7312.30 | 12 | 32.68 ± 0.83 | none | No tumors |
| | | | n# = 12 | | |
| KrasL/+; p53L/+; PtenL/+ | 9.70 | 2 | n/a | none | |
| KrasL/+; p53L/+; PtenL/L | 1.6-3.70 | 9 | 423.96 ± 73.058 | none | Large cells/nuclei; very |
| | | | n# = 6 | | proliferative, invading muscle |
| | | | | | layer, epithelial tumors |
| KrasL/+; p53L/L; PtenL/L | 0.80-2.0 | 10 | 112.35 ± 21.5 | 1/10 | Large tumors, die rapidly, |
| | | | n# = 6 | | intestinal bloating |

Further comparison of survival curves focused on double mutant mice, specifically, p53^{LoxP/LoxP}; Pten^{LoxP/LoxP} and p53^{LoxP/LoxP}; Rb^{LoxP/LoxP} since we found that mice with loss of both p53 and Pten develop invasive bladder cancer and p53 and Rb mutations have been shown to be involved in invasive bladder cancer. Calculating survival curves with an endpoint of 6 months after injection showed that all p53^{LoxP/LoxP}; Rb^{LoxP/LoxP} survived up to that time (and longer FIG. 6) and p53^{LoxP/LoxP}; Pten^{LoxP/LoxP} succumbed to large tumors from 2.5 to 6 months, again results were highly reproducible (P<0.0001).

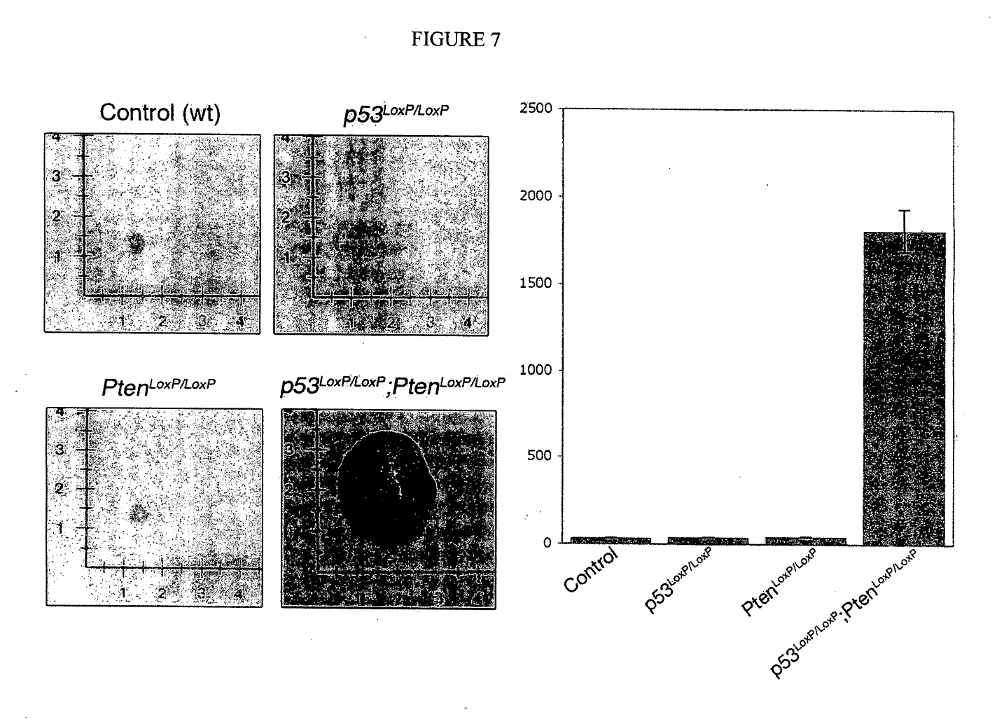

To begin analysis of the bladder tumors from the p53^{LoxP/LoxP}; Pten^{LoxP/LoxP} mice, the weights and gross anatomy of bladders and/or bladder tumors were compared between control, single mutant (p53^{LoxP/LoxP} and Pten^{LoxP/LoxP}) and the double mutant mice (p53^{LoxP/LoxP}; Pten^{LoxP/LoxP}) (FIG. 7). The gross anatomy and weights of the control and single mutant mice were all very similar (control 36.14 mg, p53^{LoxP/LoxP} 40.64 mg, Pten^{LoxP/LoxP} 43.84 mg).

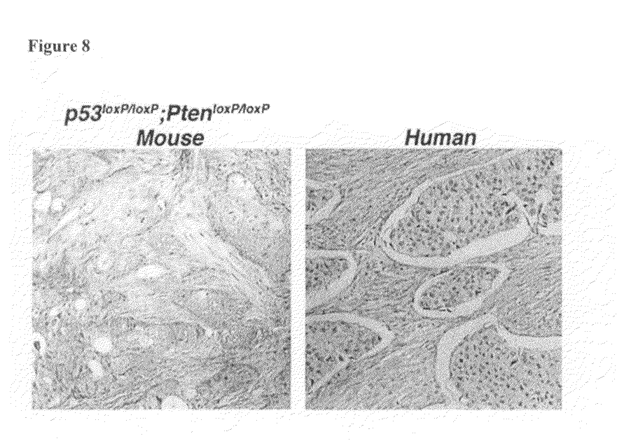

At approximately 2.2 months after injection, the histology of P53^{LoxP/LoxP}; Pten^{LoxP/LoxP} mice revealed the presence of an increase in epithelial cell layers (n=3). Also the earliest case of invasive bladder cancer was at 2.3 months after injection. After this time point the percentage of mice with bladder cancer increased (survival curve) and the average weight of a control bladder weight to the average weight of a bladder tumor (2.5-6 months after injection) was approximately 50 fold higher (FIG. 7). The histology of the bladder tumors showed the presence of an increase cytoplasmic to nuclear ratio and an increase mitotic index, which are common to various tumors, but the bladder tumors also had characteristic human bladder tumor histology such as epithelial nests (FIG. 8).

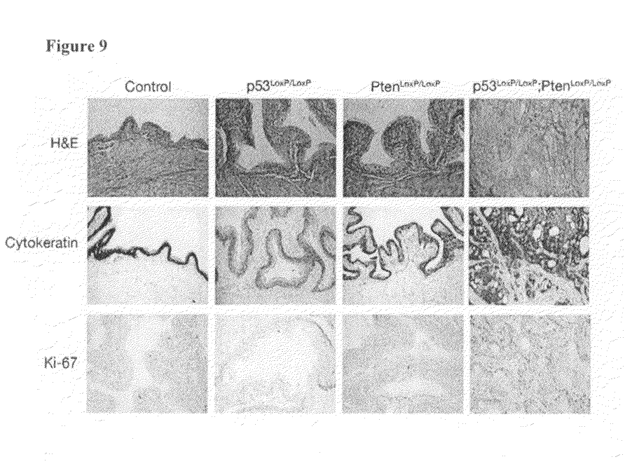

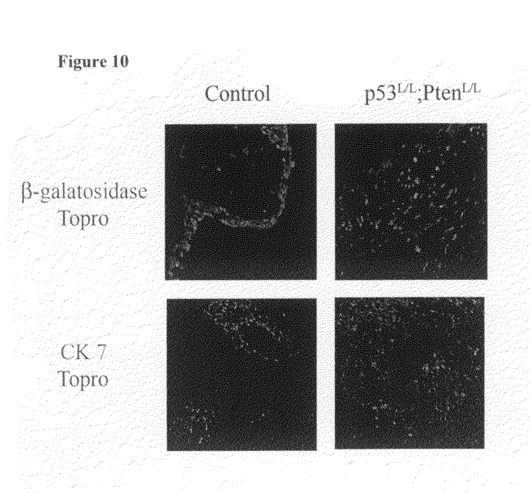

Marker analysis revealed that these tumors retained cytokeratin expression, which is also expressed in the normal urothelium and indicative that these were epithelial tumors. Also there was an increase in Ki-67 staining illustrating that the tumors are highly proliferative (FIG. 9) but also contain cells that are apoptotic (FIG. 10). These tumors were not muscle derived as shown by actin staining which stained the muscle layer that was being invaded but not the tumor cells; control shows actin staining in the muscle and not uroepithelium.

The most common site of metastasis of TCC is to the regional lymph nodes (78%) with other common sites being the liver (38%), lung (36%), bone (27%), adrenal gland (21%), and intestine (13%) and less common being the heart, brain, kidney, spleen, pancreas, meninges, uterus, ovary and prostate. Metastases were also observed at the time of dissection of the p53^{LoxP/LoxP}; Pten^{LoxP/LoxP} mice in approximately 50% of late cases (None 0.0-2.5 mos, 2/8 2.5-4.0 mos, 5/10 4.0-6.0 mos). Visible metastases were observed in the liver, intestine/intestinal lining, spleen, and diaphragm with enlarged prostatic lymph nodes and aggregated lymph nodes (intestine). Micrometastses have been shown in the liver, aggregated lymph nodes, and lung using cytokeratin 7 staining (FIG. 5) in animals that had visible metastasis in other organs.

Role of p53 and Pten in the suppression of invasive bladder cancer. p53^{LoxP/LoxP} and Pten^{LoxP/LoxP} single mutant mice did not develop tumors up to 13 months after injection (Table 1). Therefore, the mechanism by which loss of either of these two proteins still suppresses the initiation of cancer was investigated by analysis of downstream targets and markers of different mechanisms.

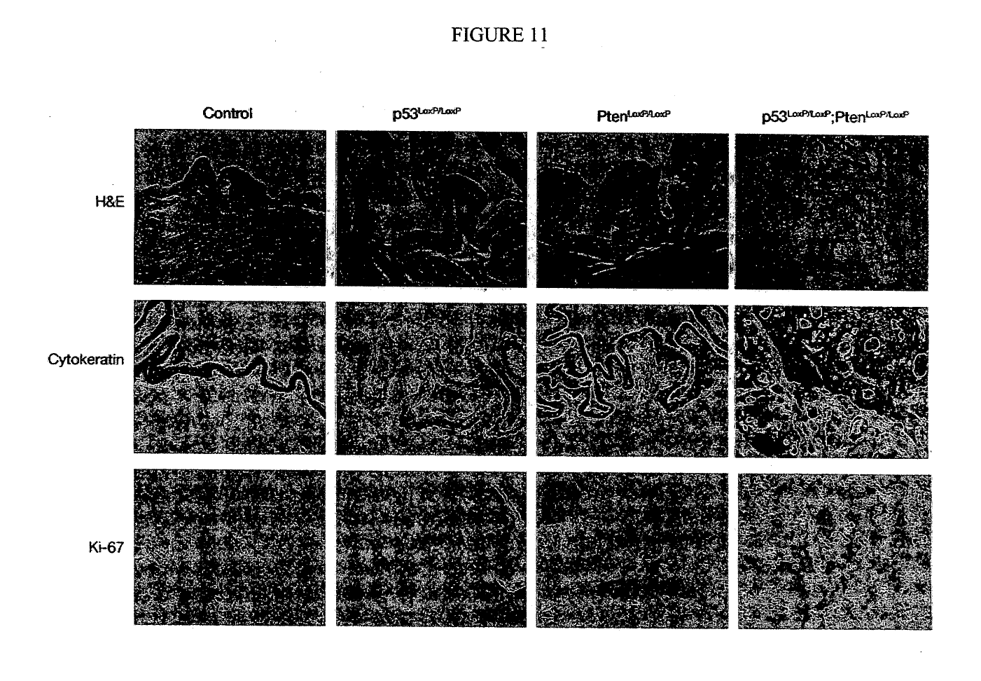

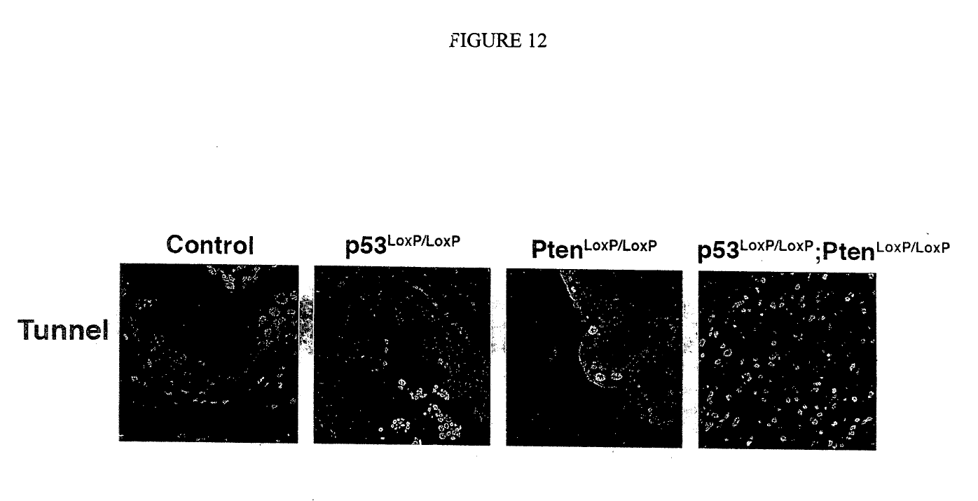

Initially, we examined pathways downstream of Pten that are commonly deregulated by loss of Pten such as pAKT^{Ser473} which is involved in survival and apoptosis and negatively regulated by Pten and pS6^{Ser235/236} which is downstream of Akt and is involved in activating translation of proteins involved in cell cycle progression and the translational machinery were examined. There were a few uroepithelial cells that were positive for PAKT^{Ser473} and PS6^{Ser235/236} in the Pten^{LoxP/LoxP} single mutant mice and not in the p53^{LoxP/LoxP} or control mice. Since there were only a limited number of cells with expression of pAKT^{Ser473} or, lead to the investigation of other pathways that might be activated and explain the suppression of tumorigenesis in these mice. Further investigation revealed that p16^{INK4a} levels were elevated in the Pten^{LoxP/LoxP} single mutant mice indicating a potential role for cellular senescence (FIG. 11). The p16^{INK4a} positive cells in the Pten^{Loxp/LoxP} single mutant mice were in the different uroepithelial cell layers (basal, intermediate, and superficial) as compared to the control and p53^{LoxP/LoxP} were a few positive p16^{INK4a} cells were only found in the superficial layer. Also the p16^{INK4a} staining pattern is slightly different between the Pten^{LoxP/LoxP} versus p53^{LoxP/LoxP}; Pten^{LoxP/LoxP} tumors, cytoplasmic versus nuclear respectively, which could indicate the specific mechanism by which p16^{INK4a} is functioning. Several proteins have also been implicated in the activation of cellular senescence such as p53 and Rb with markers of senescence being expression of p16, p21, H3K9 and SA-βgalactosidase. Interesting, p21 expression was reduced in these mice compared to controls, which would indicate that p21 is not responsible for the suppression of tumorigenesis. Expression of p53 by immunohistochemistry was hard to detect even in control mice due to low basal levels, therefore proteins downstream such as p21 were examined in p53^{LoxP/LoxP} single mutant mice. There was an increase in the number of cells with p21 expression in these mice compared to the control mice. Induction of p21 in a p53-deficient context can mean that the cells are undergoing growth arrest (Macleod et al., 1995). However, there was also can increase of apoptotic cells as shown by tunnel staining in the P53^{LoxP/LoxP} mice compared to control and Pten^{LoxP/LoxP} mice (FIG. 12), which correlates with the decrease in proliferation as seen by Ki-67 staining (FIG. 9). Therefore there is most likely a balance between cycle arrest and apoptosis that explains the block to tumorigenesis in the p53^{LoxP/LoxP} mice.

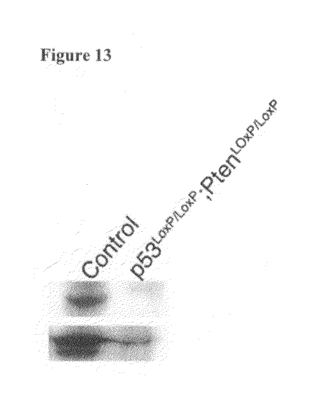

Cooperativity of p53 and Pten in invasive bladder cancer. None of the control or single mutant mice develop invasive bladder tumors or even hyperplasia, which might be explained by activation of a senescence program in the Pten^{LoxP/LoxP} mice or growth arrest and apoptosis in the p53^{LoxP/LoxP} mice. However, inactivation of p53 and Pten together, results in invasive bladder tumors. Western blot analysis of the p53 and Pten proteins revealed that both proteins are in fact down in the tumors compared to normal bladder (FIG. 13).

These tumors have an activation of the Akt pathway as shown by the expression of pAkt^{Ser473} and pS6^{Ser235/236}, which would indicate that Pten is lost in these tumors (FIG. 11). Other downstream targets of Akt, such as the TSC proteins, pS6K^{Ser2448} and p-mTOR^{Thr421/424}, which are involved in cell cycle regulation and apoptosis are also upregulated in these tumors. IHC analysis reveals that pS6K^{Ser2448} and p-mTOR^{Thr421/424} are localized in the nucleus.

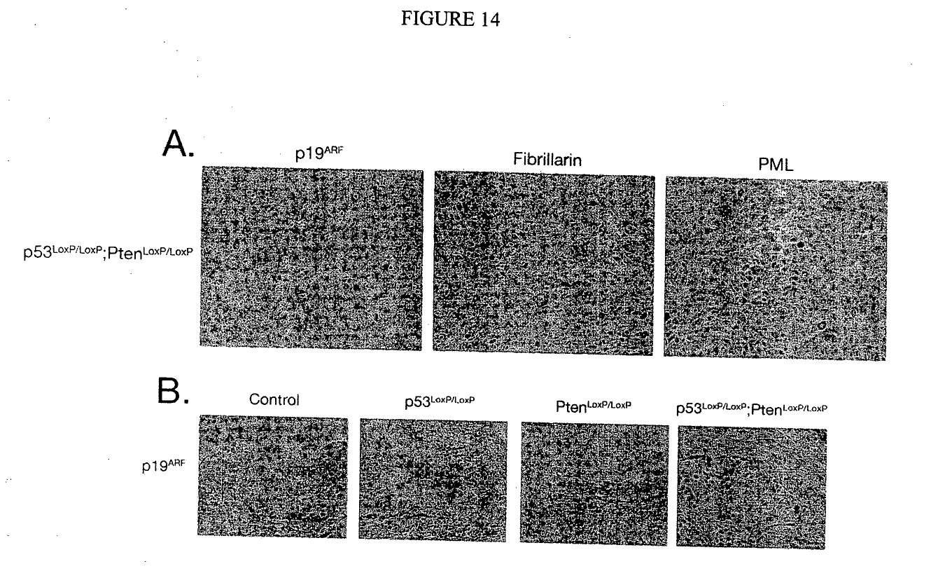

Also, activation P19^{ARF} is seen in these tumors and not in either the control or single mutant mice and the sequesterization of p19^{ARF} in the nucleoli of these tumor cells. p19^{ARF} staining is in the nucleolus and not PML bodies or nuclear speckles (not shown) as shown by the similarity to Fibrillation staining, which is a marker of nucleolus staining (FIG. 14A). p16^{INK4a} is also expressed in these tumors, which would indicate that the 9p21 locus, which is an early chromosomal target for alteration in human bladder tumors, is intact. However, even though p16^{INK4a} and p19^{ARF} are overexpressed in these tumors, it still has to be determined if the proteins are functioning properly (FIG. 14B).

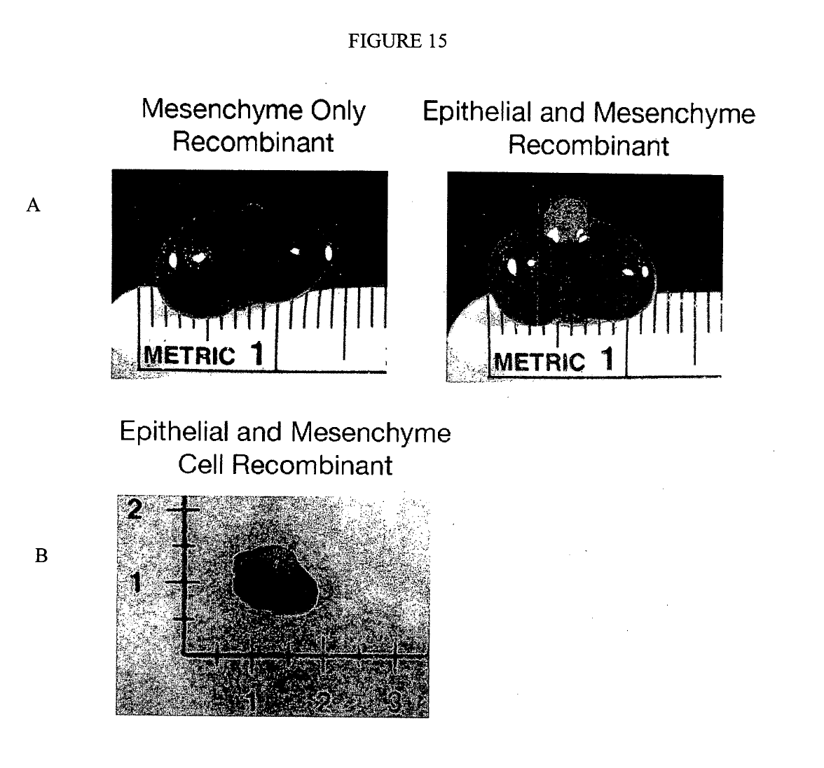

Cooperativity of p53 and Pten in invasive bladder cancer. Since we have shown that p53 and Pten cooperate to suppress invasive bladder cancer in mice, we next wanted to show that these tumor suppressors also have they same suppression in humans. Using RT4 bladder epithelial cells and 15.5 dpc rat embryonic mesenchyme, cell recombinants were set up to investigate the feasibility of using RT4 cells to form recombinants in nude mice and tissue recombinants were set up with adult rat epithelium and 15.5 dpc embryonic mesenchyme. It was found that that 1×10⁵ RT4 cells with mesenchyme from one 15.5 dpc embryonic bladder was sufficient for the formation of bladder recombinants (FIG. 15B). Use of 1.5×10⁵ RT4 cells also formed recombinants that were slightly larger. Further investigations will use cell recombinants formed with RT4 cells that are infected with lentivirus shRNA constructs for p53, Pten and p53; Pten to investigate the in vivo consequences of these tumor suppressors in bladder tumorigenesis.

Bladder cancer is thought to arise through two pathways where one leads to a non-invasive disease characterized by carcinoma in situ and the other to a muscle invasive disease. Genetic alterations have been examined to try and make a correlation between the types of alterations and the two bladder cancer pathways. For instance, p53 and Rb have been shown to cooperate in invasive bladder cancer specifically while Hras has been shown to be involved in non-invasive bladder cancer. However, p53 and Pten have not been identified before as cooperating together in bladder cancer, even though they have been studied in other cancers. The present invention provides the first animal model of invasive bladder cancer with cooperating p53 and Pten mutations that are histologically and molecularly similar to human invasive bladder tumors.

The initial strategy in formation of a bladder cancer model was to be able to introduce somatic and random mutations in adult animals, which more closely mimics what occurs in humans. The use of conditionally inducible mice allowed for the spatially and temporal aspect of the model, while the use of adenovirus-cre allowed for the recombination of genes in approximately 10% of uroepithelial cells, specifically proliferating cells. The specific recombination of a small number of uroepithelial cells in the mouse bladder is similar to what occurs in human cancer, namely mutations in a few cells giving rise to the hyperproliferation of cells that evolves to cancer.

The goal of the study was to investigate combinations of tumor suppressors (p53, Pten, and Rb) and oncogenes (K-ras) to understand the coopertivity of different pathways in bladder cancer to gain insight into which if any contributed to the noninvasive and invasive types. p53 and Rb have been known to be involved in human invasive bladder cancer as well as other human cancers. Though using the above strategy to induce bladder cancer, p53 and Rb were not found to cooperate in invasive bladder cancer or even in preneoplastic lesions. The reason for this difference might be just the simple difference between mouse and human physiology, however the mouse and human bladder are structurally and functionally very similar which is the complete opposite of other organs such as the prostate. It also might be that in the human tumors that have p53 and Rb alterations there are other cooperating mutations that are not identified and are important for the progression of bladder cancer. New tumors suppressor genes and relevant pathways are being identified that might converge on these and other pathways, for instance XAF1 is a new tumor suppressor that affects p53 stability and is influenced by the ERK1/2 signaling pathway. It would be interesting therefore to examine several human bladder tumors that are known to have p53 and Rb mutations to determine if there are any other alterations that would allow for progression of bladder cancer.

Using the strategy of Adenovirus-cre injections into the bladder lumen, did allow for the discovery of coopertivity of the p53 and Pten tumor suppressors in invasive bladder. The bladder tumors that developed in these mice had histology that was very similar to that of human invasive bladder tumors, as observed by two independent histologists. These tumors were also highly proliferative as shown by Ki-67 staining, had tumor cells nests that are typical of human bladder tumors, and regions of evident invasion from the uroepithelial layer through the lamina propria and into the muscle. Metastases also developed in approximately 50% of the p53^{LoxP/LoxP}; Pten^{LoxP/LoxP} mice that had invasive bladder tumors. Visible metastases were seen upon dissection in the liver, spleen and intestines, which are sites of metastases in humans. Micrometastases were examined in lung, liver and regional lymph nodes but H&E staining, which revealed that these tissues also had cells that look tumorigenic though further markers studies are need to see if they are from the primary bladder tumor.

Introduction of an activatable Kras into the p53^{LoxP/LoxP}; Pten^{LoxP/LoxP} seemed to speed up progression of the bladder tumors and mice usually had to be sacrificed almost 1-2 months earlier than the p53^{LoxP/LoxP}; Pten^{LoxP/LoxP} mice due to tumor burden. There has been some evidence that Kras is also involved in the progression of bladder tumors though Hras has been implicated as the predominate ras gene involved in noninvasive bladder cancer. The accelerated progression in the Kras^{LoxP/+}; p53^{LoxP/+(LoxP/LoxP)}; Pten^{LoxP/LoxP} might be a result of multiple pathways being affected at one time, namely the Erk pathway, Akt pathway, and the p53 pathway, however further research of an exact mechanism is needed.

The relevance of studying p53 and Pten in a mouse model of human cancer comes from the investigations in the Cordon-Cardo laboratory that the p53 and Akt pathways do cooperate in human invasive bladder cancer (unpublished results). Using a cohort of 165 bladder cancer patients, they conducted a study to characterize the expression patterns of Pten and phosphorylated Akt (serine 473) and to correlate that to the p53 status as well as clinicopathologic variable such as patient survival. On its own Pten immunoreactivity was reduced in advanced bladder tumors and associated with increased tumor aggressiveness and a decrease in overall survival, this was also the case for pAkt^{Ser473} positive immunoreactivity. When examined together with p53 status, it was found that there was a negative cooperative tumor suppressor effect. Alteration in Pten status with mutation in the TP53 gene was associated with tumor progression and decreased survival, specifically a difference of 6 months versus 6 years, altered Pten expression versus normal Pten expression respectively.

p53 and Pten alterations have been shown to cooperate in different cancers (Chen et al., 2005; Singh et al., 2002). Likewise p53 has been reported to regulate the transcription of Pten and PIK3CA, where p53 mutations would not enable the transcription of Pten and therefore the Akt would be activated (Stambolic et al., 2001). An activated Akt pathway would then be able to promote cell proliferation and tumorigenesis.

However further evidence of coopertivity between p53 and Pten comes from the analysis of the single mutant mice, p53^{LoxP/LoxP} and Pten^{LoxP/LoxP}. Neither of these mutant mice developed bladder cancer up to 13 months after Adenovirus-cre injection. Examining the histology of these bladders did reveal and increase of inflammation compared to control mice, which might be solely due to the deletion of either tumor suppressor. It is probably not a result of the adenovirus because the control mice did not have an inflammation response. Also there was an overall decrease in proliferation in the p53^{LoxP/LoxP} and Pten^{LoxP/LoxP} as seen by Ki-67 compared to control mice though it was more pronounced in the p53^{LoxP/LoxP} single mutant mice. Deletions of p53 usually result in cell cycle arrest or apoptosis, which would explain the decrease in proliferation and apoptosis was increased in the p53^{LoxP/LoxP} single mutants as shown by Tunnel assay. Pten deletions can result in cell proliferation do to the activation of the Akt pathway, though pAkt^{Ser473} expression in not seen in these mice. Therefore deletion of Pten, and the decrease in proliferation, might be attributed to the activation of cellular senescence. Pten inactivation has been reported to induce growth arrest through the p53 dependent cellular senescence pathway leading to a restriction in tumorigenesis (Chen et al., 2005). A similar p53 dependent cellular senescence pathway might also be activated in the Pten^{LoxP/LoxP} mice since there is a decrease in p21 expression as compared to control mice. Also another marker of cellular senescence, p16^{INK4a}, was also upregulated in these mice.

Therefore when p53 and Pten are inactivated together specifically in the bladder urothelium, the cell cycle arrest, apoptosis or cellular senescence must be bypassed in order for tumor initiation and progression. The Akt pathway was activated in the p53^{LoxP/LoxP}; Pten^{LoxP/LoxP} tumors as illustrated by pAkt^{Ser473} and pS6^{Ser235/236} expression, which would explain the increased proliferation.

The mechanism of p19% upregulation and nucleolar localization in p53^{LoxP/LoxP}; Pten^{LoxP/LoxP} tumors is unclear though very interesting. ARF is a key regulator of cell proliferation, cell growth, and a tumor suppressor. It is also a critical activator of the p53 pathway by associating with mdm2 to inhibit the ubiquitination, nuclear export, and degradation of p53. Consequently, ARF is elevated in cells lacking p53, however, in the bladder cancer model it is only elevated in p53 and Pten deficient bladder tumors and not in single p53 mutant mice. In addition, in the p53^{LoxP/LoxP}; Pten^{LoxP/LoxP} tumors, p19^{ARF} is localized in the nucleoli of every tumor cell. ARF is predominantly a nucleolar protein though it shuttles from the nucleolus to the nucleoplasm depending on function. B23 is an abundant nucleolar endoribonuclease that interacts with ARF to retain both of them in the nucleolus where they might influence ribosomal biogenesis and/or function. DNA damaging agents induce the redistribution of B23 and ARF to the nucleoplasm where they each bind to Mdm2 and allow the activation of the p53 pathway.

In the absence of both p53 and Mdm2, ARF is still able to suppress proliferation in mouse cells. Therefore there must be other proteins or pathways that are involved in the p53 independent pathway effects of ARF. It would seem that a protein in the Pten/Akt pathway might be the missing link since it is only when both p53 and Pten are deleted do bladder tumors arise, indicating that both p53-dependent and p53-independent pathways of ARF are effected and possibly sequestering ARF in the nucleolus (See Table).

TABLE 2
Cellular protein partners of the ARF tumor suppressor protein.

| ARF binding partner | Biological effect of ARF binding |
|---|---|
| APA-1 | No apparent effects |
| ARF-BP1/Mule 1 | Inhibition of ARF-BP1 ubiquitin ligase activity |
| B23 | Degradation of B23, inhibition of B23 shuttling |
| BCL6 | Inhibition of BCL6 transcriptional activity |
| CARF | Enhanced ARF-mediated cell cycle arrest |
| c-MYC | Inhibition of c-MYC transactivation |
| DP-1 | Inhibition of ARF-induced E2F proteolysis |
| E2F-1, -2, -3 | Degradation of E2F |
| Foxm 1b | Inhibition of Foxm 1b transactivation |
| HIF-1α | Inhibition of HIF-1α transactivation |
| Mdm2 | Inhibition of mdm2 ubiquitin ligase activity |
| MdmX | Enhanced p53 transactivation |
| Neurabin | Enhanced ARF-mediated cell cycle arrest |
| p120E4F | Enhanced ARF-mediated cell cycle arrest |
| Pex19p | Inhibition of p19ARF, pex19p does not bind |
| | human ARF |
| Tat-binding protein-1 | Induces ARF stabilization |
| Topoisomerase I | Enhanced topoisomerase I activity |
| Ubc9 | Probable involvement in p14ARF-mediated |
| | sumoylation |
| Werners helicase | Nucleolar exclusion of Werners helicase |

It is proposed that ARF's p53-independent functions might be dependent on ARF induced sumoylation (Tago et al., 2005). ARF triggers sumoylation of Mdm2 and nucleophosmin (NPM/B23) as well as other proteins and ARF mutants that are defective in binding Mdm2 and nucleophosmin or are excluded from the nucleolus and defective in sumoylation. Similarly, NPMmutants, that occur in myeloid leukemia cells, redirect p19^{ARF} to the cytoplasm, inhibit ARF-induced sumoylation and increase p53 activity (den Besten et al., 2006).

Additional embodiments of the present invention include, methods for identifying other proteins that might be affected and misregulating p19 in the p53; Pten double knockout mouse tumor comprising isolating cells from the cancerous bladder of the double knockout mouse and performing co-immunoprecipitation or similar analysis to identify p19 protein-protein interactions.

In another embodiment, the invention includes methods for screening cancer therapeutics, in vivo and/or in vivo or ex vivo. In one embodiment the invention includes methods for using the animal model for performing in vivo drug screening comprising inducing bladder cancer in an animal according to the methods of the invention and subsequently treating the animal with an effective amount of a test compound and comparing the cancer growth or progression to a control animal receiving placebo or sham treatment. In another embodiment, the invention includes methods for in vitro or ex vivo drug screening comprising inducing bladder cancer in an animal according to the methods of the invention; isolating a cancerous cell from the animal and growing the cell in culture; and treating the cultured cancer cell with an effective amount of a test compound and comparing the cancer cell's growth or viability to a control cell that received a placebo or sham treatment.

In another embodiment, the invention includes methods for identification of preventive cancer markers. Methods of this aspect of the invention include inducing bladder cancer in an animal according to the methods of the invention; isolating a cancerous cell from the animal; and performing gene or protein expression analysis, wherein increased or decreased expression of a particular gene can be associated with a disease state.

Materials and Methods

TABLE 3
Mouse strains and genotypes useful in the methods of the invention.

| Mouse | Strain | Mutation | | Obtained | |
|---|---|---|---|---|---|
| Name | Background | Type | Description | from | References |
| p53 | FVB; 129 | Targeted | This strain carries a conditional | MMHCC | (Jonkers et al., |
| floxed | | | mutation in the endogenous | | 2001; Marino et |
| | | | p53 gene (Trp53). LoxP sites | | al., 2000) |
| | | | were inserted into intron 1 and | | |
| | | | intron 10 of the p53 locus. | | |
| Pten | C57/Bl6 | Targeted | This strain carries a conditional | Hong Wu | (Lesche et al., |
| floxed | | | mutation in the endogenous | | 2002) |
| | | | Pten gene (MMAC1). LoxP | | |
| | | | sites were inserted flanking | | |
| | | | exon 5 of the Pten locus. | | |
| Rb | FVB; 129 | Targeted | This strain carries a conditional | MMHCC | (Marino et al., |
| floxed | | | mutation in the endogenous | | 2000) |
| | | | Rb1 gene. LoxP sites were | | |
| | | | inserted into introns | | |
| | | | surrounding exon 19 in the | | |
| | | | Rb1 locus. | | |
| Kras | B6; 129 | Targeted | These mice carry a latent | MMHCC | (Jackson et al., |
| LSL | | | point-mutant allele of Kras2 (K- | | 2001; Johnson |
| G12D | | | rasG12D). Cre-mediated | | et al., 2001; |
| | | | recombination leads to | | Tuveson et al., |
| | | | deletion of a transcriptional | | 2004) |
| | | | termination sequence (Lox- | | |
| | | | Stop-Lox) and expression of | | |
| | | | the oncogenic protein. | | |
| R26r | B6; 129 | Targeted | This mouse strain is a reporter | Jax Labs | (Soriano, 1999) |
| | | | strain, with Cre expression | | |
| | | | resulting in the removal of the | | |
| | | | loxP-flanked DNA segment | | |
| | | | and prevention of lacZ | | |
| | | | expression. When crossed | | |
| | | | with a Cre transgenic strain, | | |
| | | | lacZ is expressed in all | | |
| | | | cells/tissue where Cre is | | |
| | | | expressed. | | |

Mice were maintained under specific pathogen free (SPF) conditions and were housed in micro-isolator cages that were supplied with HEPA-filtered air and changed once a week. All cages, food, water and bedding were autoclaved prior to use. The mice were monitored for their health on a daily basis by vivarium and/or veterinary staff.

TABLE 4
PCR primers and conditions for mice

| Mouse | | Conditions | |
|---|---|---|---|
| Name | Primers | Temp. Time | PCR product |
| p53 | 1972: | 94° C. 3′ | Wildtype: |
| floxed | 5′CAC AAA AAC AGG TTA AAC CCA | 1 Cycle | 288 bp |
| | G3′ | 94° C. 1′ | Homozygous: |
| | (SEQ ID NO: 3) | 60° C. 2′ | 370 bp (loxP) |
| | 1973: | 72° C. 1′ | |
| | 5′AGC ACA TAG GAG GCA GAG AC3′ | 35 Cycles | |
| | (SEQ ID NO: 4) | | |
| | | 72° C. 3′ | |
| | | 1 Cycle | |
| | | 4° C. HOLD | |
| Pten | 1515: | 94° C. 2′ | Wildtype: |
| Floxed | 5′ACTCAAGGCAGGGATGAGC3′ | 1 Cycle | 900 bp |
| | (SEQ ID NO: 5) | 94° C. 30″ | Homozygous: |
| | 1516: | 64° C. 1′ | 1000 bp (loxP) |
| | 5′GTCATCTTCACTTAGCCATTGG3′ | 72° C. 1.5′ | |
| | (SEQ ID NO: 6) | 35 Cycles | |
| | | 72° C. 10′ | |
| | | 1 Cycle | |
| | | 4° C. HOLD | |
| Rb | 2034: | 94° C. 3′ | Wildtype: |
| floxed | 5′GGC GTG TGC CAT CAA TG3′ | 1 Cycle | 650 bp |
| | (SEQ ID NO: 7) | 94° C. 1′ | Homozygous: |
| | 2035: | 60° C. 2′ | 700 bp (loxP) |
| | 5′AAC TCA AGG GAG ACC TG3′ | 72° C. 1′ | |
| | (SEQ ID NO: 8) | 35 Cycles | |
| | | 72° C. 3′ | |
| | | 1 Cycle | |
| | | 4° C. HOLD | |
| Kras | 1982: | 94° C. 3′ | Wlldtype: |
| LSL | 5′GTC GAC AAG CTC ATG CGG | 1 Cycle | 500 bp |
| G12D | GTG3′ | 94° C. 1′ | Homozygous: |
| | (SEQ ID NO: 9) | 60° C. 2′ | 550 bp (loxP) |
| | 1980: | 72° C. 1′ | |
| | 5′CCT TTA CAA GCG CAC GCA GAC | 35 Cycles | |
| | TGT AGA3′ | 72° C. 3′ 1 | |
| | (SEQ ID NO: 10) | Cycle 4° C. | |
| | 1981: | HOLD | |
| | 5′AGC TAG CCA CCA TGG CTT | | |
| | GAG TAA GTC TGC A3′ | | |
| | (SEQ ID NO: 11) | | |
| R26r | 1726: | 94° C. 5′ | Wild Type: |
| | 5′GCG AAG AGT TTG TCC TCA ACC3′ | 1 Cycle | 600 bp |
| | (SEQ ID NO: 12) | 94° C. 30″ | Homozygous: |
| | 1727: | 59.5° C. 45″ | 300 bp |
| | 5′GGA GCG GGA GAA ATG GAT | 72° C. 1′ | |
| | ATG3′ | 30 Cycles | |
| | (SEQ ID NO: 13) | 72° C. 10′ | |
| | 1728: | 1 Cycle | |
| | 5′AAA GTC GCT CTG AGT TGT TAT3′ | 4° C. HOLD | |
| | (SEQ ID NO: 14) | | |

(foxed = flanked by LoxP recognition sequences).

Adenovirus was purchased from the University of Iowa's Vector Core Facility. Adenovirus was prepared by thawing 1 vial (25 ul) of 4×10¹⁰ PFU/ml titer on ice. It was then mixed with 20 ul of Dulbecco's Modified Eagle Medium high glucose (1×) liquid with L-glutamine and sodium pyruvate (D-MEM) media (Gibco 11995) and 5 ul of 80 ug/ml hexadimethrine bromide (Sigma H9268). A 1 ml Hamilton syringe with a 30 G/2 needle was used for adenovirus injection.

Mice were shaved an area of approximately 10% of the incision size in proximity to the bladder the day before surgery. At surgery they were anesthetized (0.1 ml Ketaset-Ketamine HCl Injection, 100 mg/ml, Fort Dodge Animal Health, Iowa, 0.08 ml Xyla-Ject (xylazine) Injectable, 20 mg/ml, Phoenix Pharmaceutical, Montana, 8.2 ml diH₂O) according to body weight (0.01 ml/g body weight) and under anesthesia the wound site was cleaned with 70% ethanol and Betadine. An excision of 2 cm was made in proximity to the bladder and the bladder removed manually. If the bladder was filled with urine, the urine was removed with a 1 ml syringe and 30 G ½ needle. Injection of adenovirus into the bladder was done by holding the bladder with serrated forceps and injection with a 1 ml Hamilton syringe and 30 G ½ needle filled with adenovirus mixture directly through the bladder muscle into the bladder lumen. Approximately 5-10 ul of adenovirus mixture was injected depending on bladder size. The bladder was re-inserted into the body cavity and the incision site closed by wound clips. The mice were monitored for recovery from anesthesia for 30 minutes and infection in the following days.

Tissue Recombinants. The tissue recombinant protocol is adapted from Oottamasathian et al. (Oottamasathien et al., 2006). Spraque Dawley 15.5 dpc pregnant rats were euthanized by CO₂ inhalation and the bladders removed from the adult female and embryos, followed by trypsin digestion on ice (1% trypsin Gibco 17073-016) Following trypsin digestion, the epithelium and/or mesenchyme were isolated by microdissection from the adult female and from embryos. The epithelium and mesenchyme were re-combined in culture, plated on a collagen plug (5:1 ratio of collagen to setting solution (100 ml 10×EBSS (Gibco), 2.45 g NaHCO₃, 7.5 ml 1M NaOH, 42.5 ml sterile diH₂O) and grown overnight at 37° C. On the following day, NCr nude mice were anesthetized (0.1 ml Ketaset-Ketamine HCl Injection, 100 mg/ml, Fort Dodge Animal Health, Iowa, 0.08 ml Xyla-Ject (xylazine) Injectable, 20 mg/ml, Phoenix Pharmaceutical, Montana, 8.2 ml diH₂O) per body weight (0.01 ml/g body weight). Under anesthesia, an excision of 2 cm was made in proximity to the kidney and the kidney removed manually. A 1 mm slit was made in the kidney capsule and the tissue recombinant placed inside between the kidney and kidney capsule. The kidney was re-inserted into the body cavity and the incision side closed by wound clips. The mice were monitored for recovery from anesthesia for 30 minutes and infection in the following days.

Cell Recombinants. The cell recombinant protocol is adapted from Oottamasathian et al. (Oottamasathien et al., 2006). Cell recombinants were done as tissue recombinants above except 1×10⁵ epithelial cells were recombined with mesenchyme (from 1 embryonic bladder), mixed with a 40 ul of a collagen plug (5:1 ratio of collagen to setting solution (100 ml 10×EBSS (Gibco), 2.45 g NaHCO₃, 7.5 ml 1M NaOH, 42.5 ml sterile diH2O) added and plated as a drop individually in a 6-well plate (Falcon 353846, specially coated for primary cultures). The following day the collagen plug was implanted between the kidney capsule and kidney.

Dissection of mice. All mice were euthanized by administration of CO2. This procedure follows the guidelines of the Panel on Euthanasia of the AVMA. Blood was removed via cardiac puncture and put into Mini-Collect tubes (a Capillary Blood Collection System with added EDTA Greiner Bio-One 450-403) were it was kept on ice before being spun down and the plasma removed. An incision was made across the abdomen of the mouse and organs were photographed (Canon Rebel XT with a Canon compact-macro lens EF 50 mm 1:25) in place if visible metastases were present. The bladder was then removed, photographed next to a metric ruler, the dry weight was taken and information was recorded. The bladder and any organs that were abnormal, had metastases or potential micrometastases were removed and either fixed overnight in 10% formalin (Fisher SF934), fixed in 4% paraformaldehyde/PBS (Fisher 04042-500) for 1 hour, snapfrozen in liquid nitrogen, or snapfrozen in OCT (Optimal Cutting Temperature, Tissue Tek 4583) in a 3-methylbutane/dry ice bath. Organs fixed in 10% formalin overnight were then washed twice with 1×PBS, left in 25% ethanol until being placed into the SAKURA Tissue Tek VIP5 Vacuum Infiltration Processor for further processing (25% alcohol for 20 mins., 50% alcohol for 20 mins., 70% alcohol twice for 20 mins., 95% alcohol twice for 20 mins., 100% alcohol twice for 20 mins., xylene twice for 30 mins., and paraffin twice for 1 hour) and paraffin embedded using the Leica EG 1160 embedding station. Tissues that were fixed in 4% paraformaldehyde go through a sucrose gradient from 15% to 30% to a 1:1 mixture of 30% sucrose and OCT (each step over night) and embedded in OCT and stored at −80° C.

β-Galactosidase Staining. Tissues that were fixed for 1 hour and frozen in OCT were sectioned into 12 um sections using a cyrostat and left to dry overnight at 4° C. The following day slides/sections were gently fix by adding ice cold 4% paraformaldehyde/PBS and incubated on ice for ˜10 minutes. Slides were rinsed 3× with PBST (1×PBS and 0.1% Tween-20 (Sigma P7949) for 5-10 minutes and 0.2 mL of freshly made staining solution (Final concentrations: 1M MgCl2 (1.3 mM), 50 mM Na₂HPO4/50 mM NaH2PO4 (5 mM), 25 mg/ml X-gal (1 mg/ml), 20% Nonidet (0.02%), 10% Na deoxycholate (0.01%), 1M PBS pH 7.4 (0.1M)) was added to each slide. Slides were kept in a humid chamber and left until a blue color develops at 37° C. (3 hours to overnight). Following color development, slides were fixed with 10% formalin, rinsed 2× with PBST for 5-10 minutes, and counterstained with Nuclear Fast Red (Vector H3403) for 5 seconds. Furthermore, the slides were dehydrated through an alcohol gradient (50%, 70%, 95%, 2×100%) into xylene and coverslipped with Clear Mount (Master Tech #MMCLEPT). Hematoxylin and Eosin Staining Paraffin-embedded sections were dewaxed by xylene for 3×5 min each. Frozen sections were allowed to thaw briefly for 1 hour. Slides were then rehydrated (100% ethanol 2× for 2 minutes, 95% EtOH for 2 minutes, 70% ethanol for 2 minutes, 50% ethanol for 2 minutes, Milli-Q diH2O 2× for 5 minutes). Staining was as follows: slides immersed in filtered Harris Modified Hematoxylin (Fisher SH26D-500) for ˜15 seconds, rinsed with Milli-Q diH2O until water, immersed in Eosin for 5 seconds, dehydrated in ascending alcohol solutions (95%, 2×100% for 2 mins each), cleared with xylene 3× for 5 minutes each, and coverslipped with Clear Mount.

### Immunohistochemical Staining

For paraffin-embedded sections, slides were treated sequentially in the following order in 350 ml staining dishes: Xylene 2× for 10 minutes each, 100% ethanol 2× for 2 minutes, Methanol+3% H2O2 for 20 minutes, 100% ethanol for 2 minutes, 95% EtOH for 2 minutes, 70% ethanol for 2 minutes, 50% ethanol for 2 minutes, Milli-Q diH2O 2× for 5 minutes. All antibodies used required antigen retrieval (microwave boiling method). Slides were then rinsed 2× with 1×PBST for 5 minutes (PBST=1×PBS+0.1% Tween 20) and blocked using 10% goat serum diluted in 1×PBS for at least 1 hour followed by 3 PBST washes for 5 minutes each. Primary antibodies were diluted in PBS/1% BSA and incubated overnight in a humid chamber at 4° C. See Table 5 for antibodies. Slides were washed 3×with PBST for 5 minutes each and a biotinylated secondary antibody (Vector) that targets the 1° Antibody was diluted 1:500 for anti-rabbit and 1:250 for anti-mouse in PBS/1% BSA and incubated for 60 minutes/humid chamber at room temperature. This was followed by 3 washes with PBST for 5 minutes each followed by the addition of Avidin Biotin Complex to each slide and incubation for 45 minutes in a humid chamber at room temperature. Again slides were washed 3× with PBST for 5 minutes each and chromagen substrate visualization was done using the NovaRed kit (Vector Labs SK-4800) followed by counterstaining with Hematoxylin (Fisher SH26-500D) (Harris, 1:4 diluted) for 5 seconds followed by: 2×DiH2O washes for 2 minutes, 50% ethanol for 2 minutes, 70% ethanol for 2 minutes, 95% ethanol for 2 minutes, 2×100% ethanol for 2 minutes, 3× Xylene for 5 minutes and coverslipping.

Immunoflorescence Staining using Tyramide. Frozen/OCT tissue was cut at 6-12 microns and stored at −80° C. Slides were washed 3× in PBST (PBS+0.1% Triton-X) for 5 minutes each and blocked using 0.1% blocking solution (tyramide kit) for at least one hour at room temperature in a humid chamber. Slides were then washed 3× with PBST, incubated with primary antibody diluted in blocking solution overnight in a humid chamber at 4° C. See Table 5 for antibodies. Next day slides were washed 3× with PBST, incubated with secondary antibody (AlexaFluor 488 or Texas Red conjugated anti-Rabbit IgG- From Molecular Probes, or equivalent) in PBST for 1 hour in a dark humid chamber at 37 C Wash 3× with PBST for 5 minutes in an opaque slide mailer.

Dry slides and mount with Vectashield+DAPI (Vector cat no. H-1200) or equivalent. View under the confocal microscope.

TABLE 5
Exemplary antibodies useful for performing immunohistochemical or
immunofluorescence analysis in any of the methods of the invention.

| | Antibody | Antibody | | | |
|---|---|---|---|---|---|
| Protein | Titer | Source | MW | | Product Information |
| pAktser473 | 1:50 | Mouse mAb | 60 | kDa | Cell Signaling 3787 |
| Ck7 | 1:50 | Mouse mAb | 54 | kDa | Abcam ab9021 |
| | IHC/IF | | | | |
| p21 | 1:50 | Mouse mAb | 21 | kDa | BD Pharmingen 556431 |
| p53 | 1:25 | Rabbit pAb | 53 | kDa | Santa Cruz (FL393) sc-6243 |
| Pten | 1:50 | Mouse mAb | 54 | kDa | Cell Signaling 9552 |
| Mdm2 | 1:250 | Mouse mAb | 55 & 90 | kDa | Levine 2A10 |
| pS6Ser235/236 | 1:50 | Rabbit pAb | 32 | kDa | Cell Signaling 2211 |
| p16 | 1:100 | Rabbit pAb | 16 | kDa | Santa Cruz sc-1207 |
| p19Arf | 1:125 | Rabbit pAb | 19 | kDa | Abcam ab80 |
| p27 | 1:500 | Mouse mAb | 27 | kDa | BD transduction 610241 |
| RB | 1:250 IHC | Mouse mAb | 100 | kDa | BD Phramingen 554136 |
| UroIII | 1:1 IHC | Mouse mAb | | | |

Senescence Assay. Cellular senescence was assayed using the Cellular Senescence Assay Kit from Chemicon (KAA002). The protocol is adapted from the kit protocol to use with unfixed frozen sections and 1×PBS pH 6.0. The growth media was aspirated from the cells/wells and washed once with 1×PBS pH 6.0. If tissue sections were used they were allowed to come to room temperature. A 1× working solution of Fixing Solution was made by diluting 100× Fixing Solution with 1×PBS pH 6.0.1 mL of 1× Fixing Solution was added per well or 100 ul per slide and incubated at room temperature for 10-15 minutes. The cells or slides were washed twice with 2 mL 1×PBS and 2 mL/well or 100 ul/slide of freshly prepared 1×SA-β-gal Detection Solution (example for 1 well: 200 ul Staining Solution A [from kit], 200 ul Staining Solution B [from kit], 50 ul X-Gal [from kit], 1.55 ml 1×PBS pH. 6.0) was added and incubated at 37° C. for at least 4 hours to overnight. The stained cells/sections were washed twice with 2 mL 1×PBS. For long term storage, stained cells were overlayed with 70% glycerol diluted in 1×PBS and stored at 4-8° C. Tissue sections were dehydrated through an alcohol gradient and coverslipped.

Immunoblotting. Standard SDS-polyacrylamide gel electrophoresis—gel preparation. After the separating gel has polymerized, decant the overlay, use Whatman paper to remove any moisture, prepare the stacking monomer.

TABLE 6
Exemplary ingredients for western blot.
4.0% gel, 0.125 M Tris, pH 6.8

| Reagents | Final Concentration |
|---|---|
| Milli-Q H2O | 3.075 ml |
| 0.5 M Tris-HCl, pH 6.8 | 1.25 ml |
| 20% (w/v) SDS | 0.025 ml |
| Acrylamide/Bis-acrylamide (30%/0.8% w/v) | 0.67 ml |
| 10% (w/v) ammonium persulfate | 0.025 ml |
| TEMED | 0.005 ml |
| Total Stack monomer | 5.05 ml |

20 ug of protein of sample was loaded per lane. Gels are run at a constant current of 50 mA with 1× Running Buffer, pH 8.3.

Western blots were transferred using apparatuses from Biorad. A 1× Transfer buffer was made containing 20% methanol (700 ml dH2O, 200 ml methanol, 100 ml 10× Transfer buffer). The Hybond-P membrane (Amersham Biosciences: PVDF Transfer Membrane Pack No. RPN303F) was activated by soaking it methanol; then equilibrated by soaking in 1× transfer buffer. Four squares (per gel) of Whatmann 3 mm paper were soaked in 1× transfer buffer. The gel was sandwiched in the transblot sponge-holder as follows: 2 sheets wet 3 mm paper to remove gel from plate, place on sponge (white plate), 1 sheet Hybond-P membrane on top of gel, 2 sheet wet 3 mm paper over membrane, roll with pipette to remove bubbles. The proteins were transferred for at least 2 hrs at 200 mAmps/box 100 Volts and after the transfer the membrane was blocked in PBST+5% non-fat dry milk for a least 1 hr at room temperature and then rinsed 3 times with 1×PBS+0.01% Tween 20 (PBST). The 1° Ab (diluted in 5% BSA+PBST) was added to the membrane and incubated at 4° C. overnight. Antibodies and conditions used are in Table 6. After incubation, the remaining Ab was washed with 1×PBST (rinsed twice every 15 min, for 3 rounds). The 2° Ab (diluted 1/2500 in PBST) was added to the membrane and incubated at room temperature for 1 hr and then washed with PBST (rinsed twice every 15 min, for 3 rounds). For protein detection, ECL plus Western Blotting Detection Kit (Amersham Biosciences, RPN2132) was used, mixed by the ratio: 2 ml Solution A to 50 ul Solution B.

TABLE 7
Exemplary ingredients for western blot.
Separating gel monomer in 0.375 M Tris, pH 8.8

| Reagents | Final Concentration |
|---|---|
| Milli-Q H2O | Gel percentage dependent |
| 1.5 M Tris-HCl, pH 8.8 | |
| 20% (w/v) SDS | |
| Acrylamide/Bis-acrylamide | Gel percentage dependent |
| (30%/0.8% w/v) | |
| 10% (w/v) ammonium persulfate | |
| TEMED | |
| Total monomer | 10.005 ml |

TABLE 8
Antibodies useful for performing immunoblotting analysis in any of
the methods of the invention.

| | Antibody | Antibody | Molecular | | |
|---|---|---|---|---|---|
| Protein | Titer | Source | Weight | | Product Information |
| pAktSer473 | 1:250 | Rabbit pAb | 60 | kDa | Cell Signaling 9271 |
| Ck7 | 1:100 | Mouse mAb | 54 | kDa | Abcam ab9021 |
| p21 | 1:100 | Mouse mAb | 21 | kDa | BD Pharmingen 556431 |
| p53 | 1:500 | Rabbit pAb | 53 | kDa | Santa Cruz (FL393) sc-6243 |
| Pten | 1:500 | Mouse mAb | 54 | kDa | Cell Signaling 9552 |
| Mdm2 | 1:250 | Mouse mAb | 55 & 90 | kDa | Levine 2A10 |
| pS6Ser235/236 | 1:500 | Rabbit pAb | 32 | kDa | Cell Signaling 2211 |
| p16 | 1:500 | Rabbit pAb | 16 | kDa | Santa Cruz sc-1207 |
| p19Arf | 1:500 | Rabbit pAb | 19 | kDa | Abcam ab80 |
| p27 | 1:1000 | Mouse mAb | 27 | kDa | BD transduction 610241 |
| RB | 1:250 | Mouse mAb | 100 | kDa | BD Phramingen 554136 |
| Actin | 1:3000 | Rabbit mAb | 45 | kDa | Cell Signaling 4970 |

Cell Lines and Growth Conditions. RT-4 cell were obtained from the American Type Culture Collection (ATCC catalog# HTB-2). This human cell line was derived from a transitional cell papilloma of the bladder. The growth media and conditions are as follows: McCoy's 5a Medium with 1.5 mM L-glutamine and 2.2 g/L sodium bicarbonate supplemented with 10% fetal bovine serum and cell are grown in a 5% CO2 in air atmosphere. Phoenix Ampho cells were obtained from the American Type Culture Collection (ATCC catalog# SD-3443). This human cell line is a retrovirus producer line derived from the 293T cell line (a human embryonic kidney line transformed with adenovirus E1a and carrying a temperature sensitive T antigen co-selected with neomycin). The growth media and conditions are as follows: Dulbecco's Modified Eagle Medium high glucose (1×) liquid with L-glutamine and sodium pyruvate (D-MEM) media (Gibco 11995) supplemented with 10% fetal bovine serum.

Retroviral Infection of Cell Lines. Initially, 1.2×10 Phoenix Ampho (Phoenix A) were seeded in a 100 mm dish to have a cell density of 30 to 50%. The transfection was set up according to the following 2 tables (Tables 9 and 10).

TABLE 9
DNA mixture

| | | OPTI-MEM I |
|---|---|---|
| | DNA (μg) | (Invitrogen: 11058-021) (μl) |
| 35 mm | 1 | 100 |
| 60 mm | 5 | 300 |
| 100 mm | 12 | 800 |

TABLE 10
Lipofectamine mixture

| | LIPOFECTAMINE | |
|---|---|---|
| | (Invitrogen: 18324-020) μl | OPTI-MEM I (μl) |
| 35 mm | 5 | 100 |
| 60 mm | 15 | 300 |
| 100 mm | 40 | 800 |

The lipofectamine mixture was added to DNA mixture and incubated for 20 to 40 min at room temperature. OPTI-MEM I media was then added to mixture (35 mm 0.8-ml, 60 mm 2.4 ml, 100 mm 6.4 ml). Media was removed from Phoenix cells and washed with 1×PBS. DNA/lipofectamine mixture was added to the cells and allowed it to incubate for 5 hrs at 37° C. in a CO2 incubator before fresh DMEM+10% serum was added. After 24 hours, cells were selected for transfection by adding 1× Puromycin (8 mg/ml stock solution Sigma P-8833) to the media for 3 days before allowing the Phoenix cells to recover by adding fresh DMEM+10% serum without puromycin. On the day of infection, target cells should be at a cell density of 10%-20%. For infection, media was removed from the Phoenix cells, which contains the virus, using a syringe (5 ml for 60 mm dish, 10 ml for 100 mm dish) then attached to a 0.45 um syringe filter (VWR: 28146-002) and filtered. 1× Polybrene (8 mg/ml stock solution in dH2O Hexadimethrine bromide Sigma: H-9268) was added to the filtered media containing the virus which was then added to the target cells. Infection was repeated for 3 days before fresh DMEM+10% serum was added and the target cells were allowed to recover for a day. Cells were the used for protein extraction or split for other experiments (growth assay or soft agar assay).

ShRNA-RNA knockdown. shRNA plasmids (MSCV-LMP-empty, MSCV-LMP-p53 and MSCV-LMP-Pten) were obtained from Greg Hannon's laboratory which were cloned into the XhoI and EcoRI sites (Plasmid diagram from Open Biosystems, FIG. 4). These plasmids are MSCV-based retroviral vectors that express shRNAmir from the retroviral LTR promotere. The plasmids were transfected into Phoenix A cells according to the above retroviral protocol. Stable intergrants were select using puromycin resistance and GFP serves as a marker for retroviral intergration. After transfection, the retroviruses infected the appropriate cell lines to allow for RNA knockdown. To assure that the corresponding protein levels were decreased, SDS lysates were made 72 hours after lenti-viral infection and were examined by Western blot analysis.

Growth Assay. Cells were seeded in five 6-well plate dishes (Five dishes for Day 0, 2, 4, 6, 8) so that they were a low confluency and rinsed twice with serum free RPMI media before medium with appropriate amounts of serum was added and changed every other day. One dish was fixed on day 0, 2, 4, 6, 8 with 10% formalin and kept at 4° C. before the assay. At time of the assay, the formalin was removed and cells were washed with 1×PBS three times and 0.5 ml of NBB stain solution (NBB solution (500 ml): Naphtol blue black 500 mg, Acetic acid 45 mL, Sodium acetate 4.1 g) was added per well of 6-well plate for 30 min at room temperature. The wells were washed with dH2O at least twice to wash away trace amount of NBB dye and allowed to dry in the warm room. To assay the cell growth, the dye was extracted with 1501 ul of 50 nM NaOH and 75 μl of solution was taken from each well and added into a 96-well plate. A microplate reader was used to read the 595 nm wavelength.

Tumorgenicity/Soft Agar Assay. Preparation of the bottom layer of 6 well plates: A 30 ml stock of 5% agarose (Agarose A or Nobe agar) in Milli-Q diH2O was prepared (melt the agarose in a microwave and put it immediately in a 56° C. water bath). The needed amount of RPMI or DMEM was pre-warmed to 56° C. and melted agarose was added to the warmed RPMI or DMEM to a final concentration of 0.5%. The RPMI or DMEM/agarose mix was incubated at 37° C. for about 4-5 minutes before use and serum, pen/strep, glutamine, HEPES buffer, non-essential amino acid and any other growth factors were added as necessary to the RPMI or DMEM/agarose mix and distributed 2 ml per well. It solidified for about 4-5 minutes at room temperature.

Preparation of cells and top layer of 6 well plates: Individual tubes were prepared for each cell type so that there are 100 cells/well with 0.3 ml (RPMI or DMEM+serum) and enough cells to do the experiment 4× and kept in a 37° C. water bath. Melted agarose was added to pre-warmed RPMI or DMEM (calculated about 3.7 ml/4 wells) to get a final agarose concentration of 0.35% in the well. About 4-5 minutes before use, serum, pen/strep, glutamine, and any other necessary growth factors/reagents were added to the RPMI or DMEM/agarose at 37° C. and 3.7 ml of RPMI or DMEM/agarose mix was distributed per tube of 0.3 ml of cells and 1 ml of the resulting mixture was added on top of the bottom layer in each well. It was allowed to solidify at room temperature for 15-20 minutes before being put in the CO2 incubator. Growth media (0.1-0.2 ml) was added every 2-3 days to keep the plates moist. The colonies were visible in 8-10 days. The colonies were counted after staining with piododnitrotetrazolium violet (Sigma 1-8377) in DMSO (dilute 1/100 in PBS), which was added to the top agar of the 6-well plate and incubated overnight in the CO2 incubator. Colonies were counted on the next day.

Statistical Analysis. Kaplan-Meier survival curves were calculated using Graphpad Prism from Graphpad Software, graphpadsoftware.com

It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present invention will be apparent from the appended claims.

1. A knockout animal model comprising a transgenic animal wherein the genome of at least one cell of the transgenic animal contains a disruption in at least one allele of its p53 genes and at least one allele of its Pten genes, and wherein the disruption prevents the expression of a functional protein.

2. The animal model of claim 1, wherein the disruption results in the cell expressing reduced levels of p53 and Pten as compared to wild-type.

3. The animal model of claim 2, wherein the disease is a bladder disease and the cell is a bladder cell.

4. The animal model of claim 1, wherein the disruption results from Cre recombinase catalyzed recombination of a LoxP sequence existing in the endogenous p53 and Pten genes of the transgenic animal.

5. The animal model of claim 1, wherein the animal is a mouse.

6. A method for producing a transgenic double knockout animal exhibiting decreased levels of p53 and Pten relative to a wild-type animal, said method comprising:
(a) providing a transgenic animal having whose genome comprises at least one LoxP sequence inserted into at least one allele of a p53 gene, and at least one allele of a Pten gene;
(b) injecting a vector comprising adenovirus-Cre recombinase into one or more tissues of the animal, wherein at least one transfected somatic cell of the animal expresses the Cre recombinase resulting in a disruption in at least one allele of a p53 gene, and at least one allele of a Pten gene.

7. The method of claim 6, wherein the animal is a mouse.

8. A transgenic double knockout mouse produced by the method of claim 7, wherein the genome of at least one cell of the transgenic mouse comprises a disruption of at least one allele of the endogenous p53 and Pten genes, and wherein said disruption results in the transgenic double knockout mouse exhibiting decreased levels of p53 and Pten relative to a wild-type mouse.

9. Cells derived from the animal model of claim 1.

10. The cells of claim 9, wherein the cells are cultured in vitro.

11. A method for screening a test compound for anti-cancer activity in a double knockout animal, comprising: a) administering said test compound to an animal having at least one cell comprising a disruption in an endogenous p53 and Pten gene; b) assaying the animal for the development of bladder cancer; and c) comparing the assay in the knockout animal with the same assay carried out in control animals which have not been administered the test compound by monitoring for the prevention of the development of cancerous tissue from precancerous tissue or the amelioration of the malignant cancerous tissue, thereby screening the test compound for anti-cancer activity.

12. A method of identifying markers associated with bladder disease or bladder cancer, the method comprising comparing the presence, absence or level of expression of at least one gene or protein in a cell whose genome comprises a disruption of at least one allele of both the endogenous p53 and Pten genes with the level or expression of the gene or protein in a second animal, wherein the second animal has the same genetic background as the first animal but does not comprise a disruption of the endogenous p53 and Pten genes, wherein the difference between the first transgenic animal and the second animal in the presence, absence or level of expression of the gene or protein indicates that the expression of the gene is a marker associated with cancer of the bladder.

13. The method of claim 12, wherein the animal is a mouse.
